(19) United States
(12) Patent Application Publication
(10) Pub. No.: US 2026/0151631 A1
(43) Pub. Date: Jun. 4, 2026

(54) MODIFIED HIGH FREQUENCY NEUROMODULATION SIGNALS, AND ASSOCIATED SYSTEMS AND METHODS

(71) Applicant: Nevro Corp., Redwood City, CA (US)

(72) Inventors: Dongchul Lee, Agua Dulce, CA (US); Kerry Bradley, Glendale, CA (US)

(21) Appl. No.: 19/465,612

(22) Filed: Jan. 30, 2026

Related U.S. Application Data
(62) Division of application No. 17/824,737, filed on May 25, 2022, now Pat. No. 12,558,547.
(60) Provisional application No. 63/192,998, filed on May 25, 2021.

(51) Int. Cl. A61N 1/36

(52) U.S. Cl. CPC A61N 1/36062; A61N 1/36171; A61N 1/36175

(57) ABSTRACT

The present technology is directed generally to spinal cord modulation and associated systems and methods for treating pain and other patient conditions. In particular, the present technology includes modified high frequency neuromodulation signals and administration patterns.

### CROSS-REFERENCE TO RELATED APPLICATION(S)

The present application is a divisional application of U.S. Ser. No. 17/824,737, filed on May 25, 2022, which claims priority to U.S. Provisional Application No. 63/192,998, filed May 25, 2021, the disclosures of all of which are incorporated by reference herein in their entireties.

### TECHNICAL FIELD

The present technology is directed generally to spinal cord modulation and associated systems and methods for treating patient conditions, including modified high frequency neuromodulation signals and administration patterns.

### BACKGROUND

Neurological stimulators have been developed to treat pain, movement disorders, functional disorders, spasticity, cancer, cardiac disorders, and various other medical conditions. Implantable neurological stimulation systems generally have an implantable pulse generator and one or more leads that deliver electrical pulses to neurological tissue or muscle tissue. For example, several neurological stimulation systems for spinal cord stimulation (“SCS”) have cylindrical leads that include a lead body with a circular cross-sectional shape and one or more conductive rings spaced apart from each other at the distal end of the lead body. The conductive rings operate as individual electrodes and, in many cases, the SCS leads are implanted percutaneously through a needle inserted into the epidural space, with or without the assistance of a stylet. Once implanted, the pulse generator applies electrical pulses to the electrodes, which in turn modify the function of the patient's nervous system, such as by altering the patient's responsiveness to sensory stimuli and/or altering the patient's motor-circuit output.

In the context of treating pain, electrical signals have been applied at low frequencies (e.g., frequencies less than 1.2 kHz) and high frequencies (e.g., frequencies greater than 1.2 kHz). When the electrical pulses are applied at a low frequency (e.g., less than 1.2 kHz), the signals can generate sensations that mask or otherwise alter the patient's sensation of pain. For example, in many cases, patients report a tingling or paresthesia that is perceived as more pleasant and/or less uncomfortable than the underlying pain sensation. While this may be the case for many patients, many other patients may report less beneficial effects and/or results. When the electrical pulses are applied at a high frequency (e.g., greater than 1.2 kHz), the signals generally do not generate paresthesia and produce generally effective pain relief. However, the onset of pain relief in response to high frequency signals is often delayed, leading to a period of several hours to several days during which the patient is still suffering from pain. Accordingly, there remains a need for improved techniques and systems for addressing patient pain.

### BRIEF DESCRIPTION OF THE DRAWINGS

FIG. 1A is a partially schematic illustration of an implantable spinal cord modulation system positioned at a patient's spine to deliver therapeutic signals in accordance with some embodiments of the present technology.

FIG. 1B is a partially schematic, cross-sectional illustration of a patient's spine, illustrating representative locations for implanted lead bodies in accordance with some embodiments of the present technology.

FIG. 2 is a schematic illustration of a representative lead body suitable for providing modulation to a patient in accordance with several embodiments of the present technology.

FIG. 3 is a schematic illustration of an existing high frequency electrical therapy signal.

FIGS. 4A-4C are schematic illustrations of modified high frequency electrical therapy signals having asymmetric bi-phasic pulses in accordance with embodiments of the present technology.

FIGS. 5A-5C are schematic illustrations of additional modified high frequency electrical therapy signals having relatively long interphase intervals in accordance with embodiments of the present technology.

FIGS. 6A-6C are partially schematic illustrations of a pattern for administering an electrical therapy signal in accordance with embodiments of the present technology.

FIG. 7 is a line graph illustrating the relative firing rate of excitatory neurons versus inhibitory neurons in response to select high frequency electrical therapy signals.

FIGS. 8A and 8B are bar graphs illustrating the relative firing rate of excitatory and inhibitory neurons in response to high frequency electrical therapy signals having asymmetric bi-phasic pulses, and FIG. 8C is a line graph illustrating the relative firing rate of excitatory and inhibitory neurons in response to high frequency electrical therapy signals having asymmetric bi-phasic pulses.

FIG. 9 is a bar graph illustrating the relative firing rate of neurons in response to high frequency electrical therapy signals having relatively long interphase intervals.

FIG. 10 is a graph comparing pain relief achieved using various high frequency electrical therapy signals described herein.

FIG. 11 is a graph demonstrating the expected pain relief over time of existing and modified high frequency electrical therapy signals.

### DETAILED DESCRIPTION

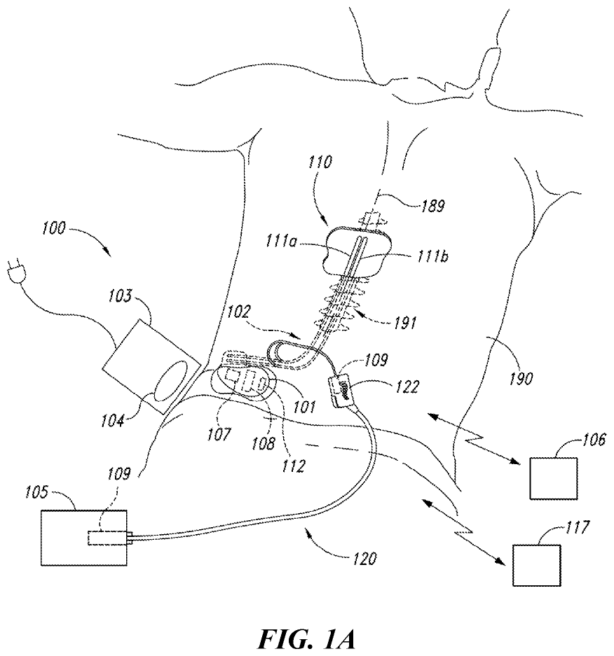

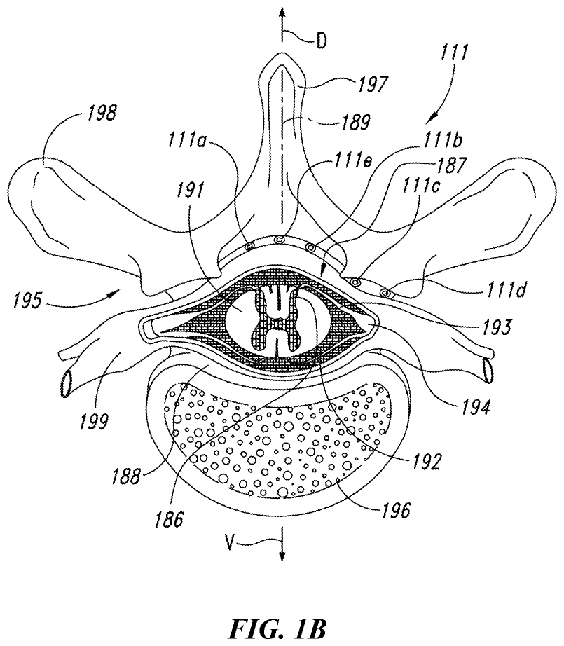

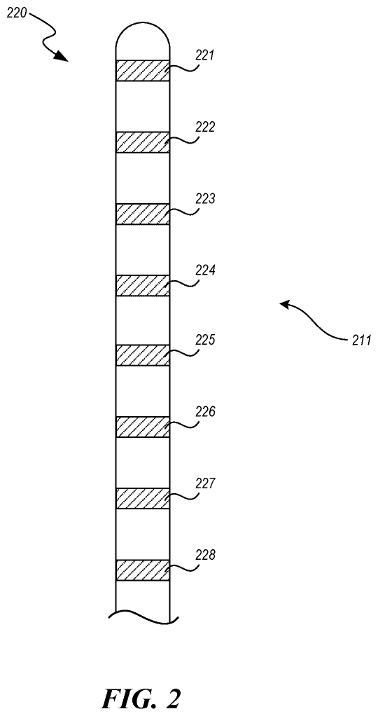

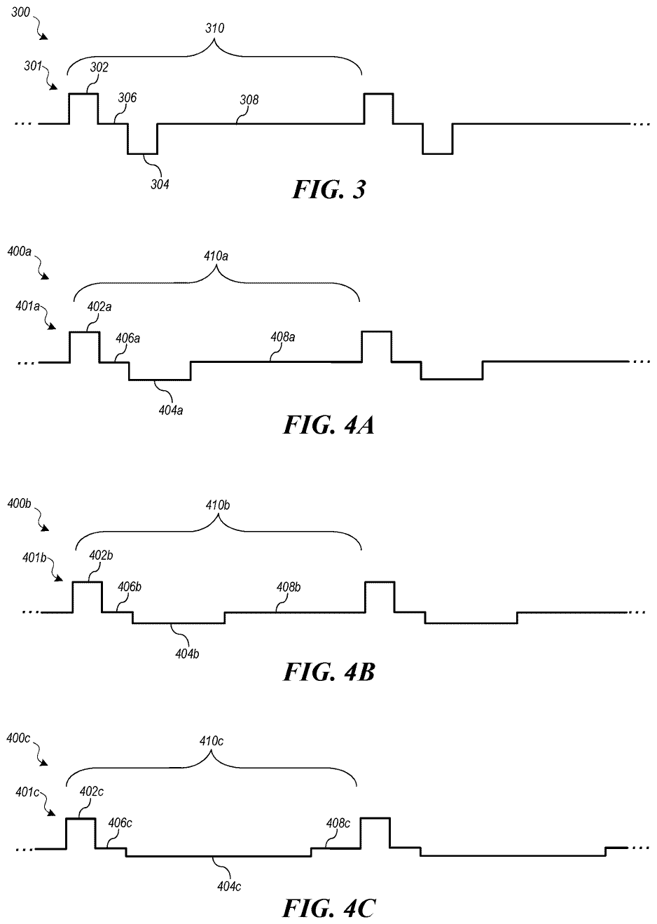

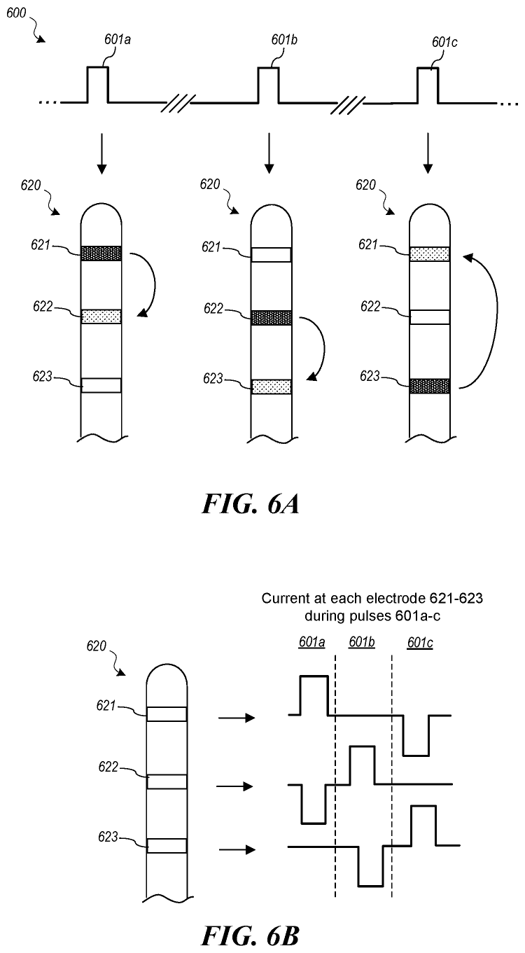

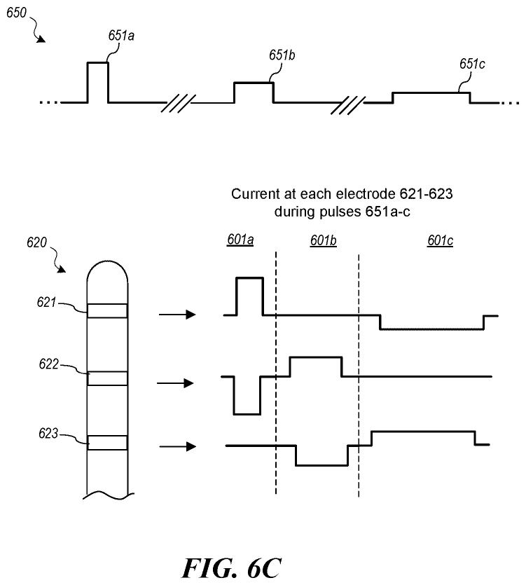

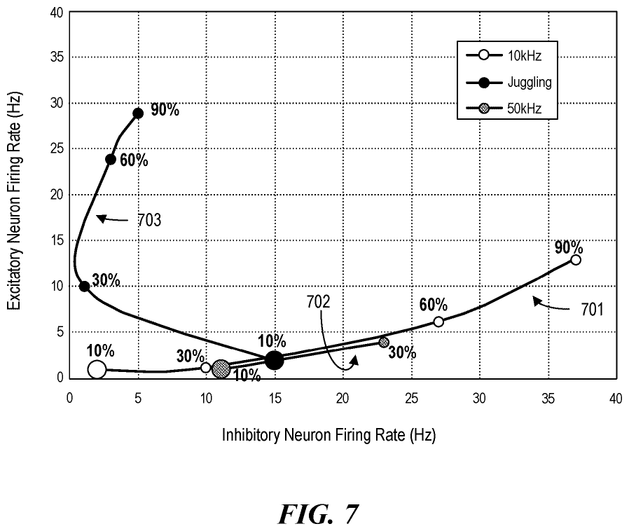

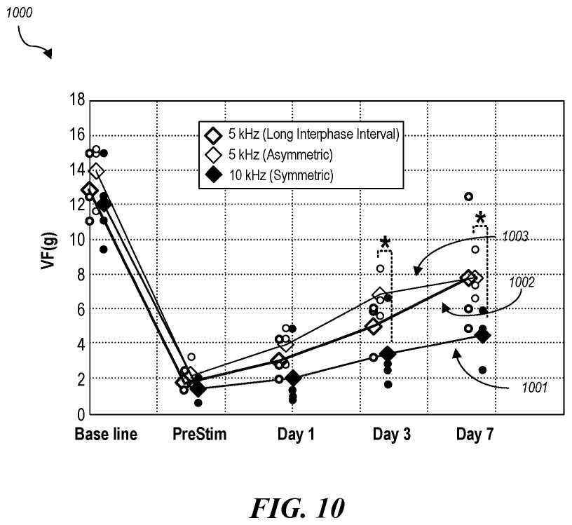

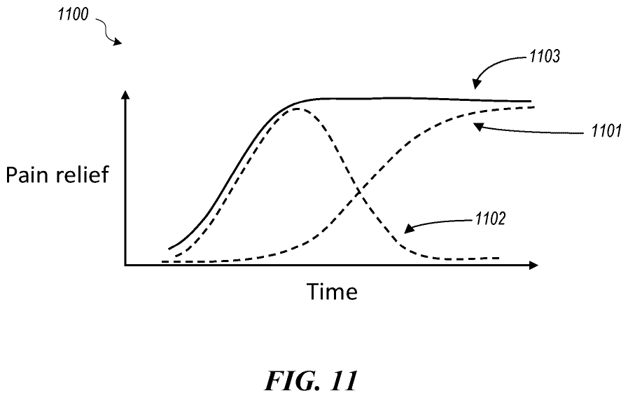

This detailed description includes the following headers and sections, which are provided for convenience only and do not interpret the scope or meaning of the claimed present technology:
Definitions of selected terms are provided under Heading 1.0 (“Definitions”);
General aspects of the present technology are described below under Heading 2.0 (“Overview of Present Technology”);
Representative treatment systems and their characteristics are described under Heading 3.0 (“System Characteristics”) with reference to FIGS. 1A, 1B and 2;
Representative electrical therapy signals for use in treating patients are described under Heading 4.0 (“Representative Therapy Signals”) with reference to FIGS. 3A-5;
Representative patterns for administering electrical therapy signals for use in treating patients are described under Heading 5.0 (“Representative Patterns of Administration”) with reference to FIGS. 6A-6C;
Potential effects of the representative therapy signals and patterns of administration are described under Heading 6.0 (“Selectivity of Modified High Frequency Electrical Therapy Signals”) with reference to FIGS. 7-9;
Therapies combining existing high frequency signals with modified high frequency signals are described under Heading 7.0 (“Combined Therapy”) with reference to FIGS. 10 and 11;
Additional expected benefits of certain embodiments of the present technology are described under Heading 8.0 (“Additional Expected Benefits Associated with Certain Embodiments”); and
Representative examples are described under Heading 9.0 (“Representative Examples”).

### 1.0 Definitions

Unless otherwise stated, the terms “generally,” “about,” and “approximately” refer to values within 10% of a stated value. For example, the use of the term “about 100” refers to a range of 90 to 110, inclusive. In instances where relative terminology is used in reference to something that does not include a numerical value, the terms are given their ordinary meaning to one skilled in the art.

As used herein, and unless otherwise noted, the terms “modulate,” “modulation,” “stimulate,” and “stimulation” refer generally to signals that have an inhibitory, excitatory, and/or other effect on a target neural population. Accordingly, a spinal cord “stimulator” can have an inhibitory effect on certain neural populations, an excitatory effect on certain neural populations, and/or both an inhibitory and excitatory effect on certain neural populations.

As used herein, the terms “neuromodulation signal”, “electrical therapy signal,” “electrical signal,” and “therapy signal” and other associated terms are used interchangeably and generally refer to an electrical signal that can be characterized by one more parameters, such as, frequency, pulse width, and/or amplitude.

As used herein, the term “existing high frequency neuromodulation signals,” “existing high frequency signals,” or the like generally refer to an electrical signal having symmetrical bi-phasic pulses having a relatively short interphase interval of about 20 microseconds or less and repeating at a frequency between about 1.2 kHz and about 100 KHz. An example of an existing high frequency neuromodulation signal is illustrated in FIG. 3.

As used herein, the terms “modified high frequency neuromodulation signals,” “modified high frequency signals,” or the like generally refer to an electrical signal that (a) has asymmetric bi-phasic pulses, (b) has relatively long interphase intervals, and/or (c) is administered across multiple different pairs of electrodes (also referred to herein as a “juggling pattern”). Examples of modified high frequency neuromodulation signals are illustrated in FIGS. 4A-6C.

As used herein, the term “pulse width” refers to the width of any phase of a repeating pulse, such as the portion of a pulse at a given polarity, unless explicitly described otherwise. For example, the use of the term pulse width with respect to a signal having bi-phasic pulses can refer to the duration of an anodic pulse phase or a cathodic pulse phase. The use of the term pulse width with respect to a signal having monophasic pulses can refer to the duration of the monophasic pulse phase.

As used herein, the use of the terms “delivery,” “deliver,” and variants thereof while describing the relationship between an electrical contact and the phase of a bi-phasic signal “delivered by” the contact describes the phase of the contact during administration of the bi-phasic pulse, and does not necessarily indicate current is flowing out of the contact. For example, if a first contact is said to “deliver” an anodic pulse phase of a bi-phasic pulse, the first contact is acting as an anode during delivery of the bi-phasic pulse. Similarly, if a second contact is said to “deliver” a cathodic pulse phase of the bi-phasic pulse, the second contact is acting as a cathode during delivery of the bi-phasic pulse.

As used herein, “proximate a spinal cord region” refers to the placement of a signal delivery element such that it can deliver electrical stimulation to a neural population associated with the spinal cord or associated nervous system structures. For example, “proximate a spinal cord region” includes, but is not limited to, the relative lead positions described and shown in FIG. 1B, as well as other positions not expressly described herein.

### 2.0 Overview of the Present Technology

The present technology is generally directed to spinal cord modulation and associated systems and methods for treating pain and other patient conditions. In particular, the present technology includes modified high frequency neuromodulation signals and administration patterns. For example, in some embodiments the present technology includes modified high frequency electrical therapy signals having asymmetric bi-phasic pulses. The bi-phasic pulses generally have an anodic pulse phase and a cathodic pulse phase. The anodic pulse phase and the cathodic pulse phase can have different pulse widths. For example, the cathodic pulse phase can have a greater pulse width than the anodic pulse phase. To ensure the signal is nevertheless charge balanced, the amplitude of the anodic pulse phase and the cathodic pulse phase can also be different. For example, in the foregoing example in which the cathodic pulse phase has a greater pulse width than the anodic pulse phase, the cathodic pulse phase can have a smaller amplitude than the anodic pulse phase to ensure that the total charge delivered during the bi-phasic pulse is equal and opposite.

In some embodiments, the present technology includes modified high frequency electrical therapy signals having relatively long interphase intervals between paired anodic and cathodic pulse phases. For example, the interphase interval can have a duration that is longer than the pulse width of the anodic pulse phase and/or the cathodic pulse phase. In some embodiments, the present technology includes modified high frequency electrical therapy signals administered across multiple different pairs of electrodes, such that each pulse of the signal is delivered via a pair of contacts that is different than the pair of contacts used to deliver the immediately preceding pulse.

Without being bound by any particular theory, the use of modified high frequency signals may modulate a target population of neurons in a different manner than existing high frequency signals and/or low frequency signals. For example, modified high frequency signals may preferentially modulate a first set of neurons (e.g., adapting neurons, excitatory neurons, etc.) within a target set of neurons, whereas existing high frequency signals may preferentially modulate a second set of neurons (e.g., nonadapting neurons, inhibitory neurons, etc.) within the target set of neurons. In some embodiments, the modified high frequency signals may modulate both the first set of neurons and the second set of neurons, but in a different manner than existing high frequency signals. Therefore, in some embodiments, modified high frequency signals can be administered to a patient to achieve a first physiological effect or result, and existing high frequency signals can be delivered to the patient to achieve a second physiological effect or result. The first and second physiological effects can be complementary, synergistic, or antagonistic.

In some embodiments, the modified high frequency signals can induce a relatively rapid onset of pain relief in a patient. For example, the modified high frequency signals can induce pain relief within several hours of, within several minutes of, or simultaneous with the onset of signal administration. Accordingly, in some embodiments the modified high frequency signals can be delivered in combination with existing high frequency signals to provide more rapid onset of pain relief.

### 3.0 System Characteristics

FIG. 1A schematically illustrates a representative patient therapy system 100 for treating a patient's motor, sensory, and/or other functioning, arranged relative to the general anatomy of the patient's spinal column 191. The system 100 can include a signal generator 101 (e.g., an implanted or implantable pulse generator or IPG), which can be implanted subcutaneously within a patient 190 and coupled to one or more signal delivery elements or devices 110. The signal delivery elements or devices 110 can be implanted within the patient 190, at or off the patient's spinal cord midline 189. The signal delivery elements 110 carry features for delivering therapy to the patient 190 after implantation. The signal generator 101 can be connected directly to the signal delivery devices 110, or it can be coupled to the signal delivery devices 110 via a signal link, e.g., a lead extension 102. In some embodiments, the signal delivery devices 110 can include one or more elongated lead(s) or lead body or bodies 111 (identified individually as a first lead 111a and a second lead 111b). As used herein, the terms signal delivery device, signal delivery element, lead, and/or lead body include any of a number of suitable substrates and/or supporting members that carry electrodes/devices for providing therapy signals to the patient 190. For example, the lead or leads 111 can include one or more electrodes or electrical contacts that direct electrical signals into the patient's tissue, e.g., to provide for therapeutic relief. In some embodiments, the signal delivery elements 110 can include structures other than a lead body (e.g., a paddle) that also direct electrical signals and/or other types of signals to the patient 190, e.g., as disclosed in U.S. Patent Application Publication No. 2018/0256892, incorporated herein by reference in its entirety. For example, paddles can be more suitable for patients with spinal cord injuries that result in scarring or other tissue damage that impedes cylindrical leads.

In some embodiments, one signal delivery device can be implanted on one side of the spinal cord midline 189, and a second signal delivery device can be implanted on the other side of the spinal cord midline 189. For example, the first and second leads 111a, 111b shown in FIG. 1A can be positioned just off the spinal cord midline 189 (e.g., about 1 mm offset) in opposing lateral directions so that the two leads 111a, 111b are spaced apart from each other by about 2 mm. In some embodiments, the leads 111 can be implanted at a vertebral level ranging from, for example, about T4 to about T12. In some embodiments, one or more signal delivery devices can be implanted at other vertebral levels, e.g., as disclosed in U.S. Pat. No. 9,327,121, incorporated herein by reference in its entirety.

The signal generator 101 can transmit signals (e.g., electrical signals) to the signal delivery elements 110 that excite and/or suppress target nerves. The signal generator 101 can include a machine-readable (e.g., computer-readable or controller-readable) medium containing instructions for generating and transmitting suitable therapy signals, such as those described below with respect to FIGS. 3-5C. The signal generator 101 and/or other elements of the system 100 can include one or more processor(s) 107, memory unit(s) 108, and/or input/output device(s) 112. Accordingly, the process of providing modulation signals, providing guidance information for positioning the signal delivery devices 110, establishing battery charging and/or discharging parameters, and/or executing other associated functions can be performed by computer-executable instructions contained by, on, or in computer-readable media located at the pulse generator 101 and/or other system components. Further, the pulse generator 101 and/or other system components can include dedicated hardware, firmware, and/or software for executing computer-executable instructions that, when executed, perform any one or more methods, processes, and/or sub-processes described in the materials incorporated herein by reference. The dedicated hardware, firmware, and/or software also serve as “means for” performing the methods, processes, and/or sub-processes described herein. The signal generator 101 can also include multiple portions, elements, and/or subsystems (e.g., for directing signals in accordance with multiple signal delivery parameters), carried in a single housing, as shown in FIG. 1A, or in multiple housings. For example, the signal generator can include some components that are implanted (e.g., a circuit that directs signals to the signal delivery device 110), and some that are not (e.g., a power source). The computer-executable instructions can be contained on one or more media that are implanted within the patient and/or positioned external to the patient, depending on the embodiment.

The signal generator 101 can also receive and respond to an input signal received from one or more sources. The input signals can direct or influence the manner in which the therapy, charging, and/or process instructions are selected, executed, updated, and/or otherwise performed. The input signals can be received from one or more sensors (e.g., an input device 112 shown schematically in FIG. 1A for purposes of illustration) that are carried by the signal generator 101 and/or distributed outside the signal generator 101 (e.g., at other patient locations) while still communicating with the signal generator 101. The sensors and/or other input devices 112 can provide inputs that depend on or reflect patient state (e.g., patient position, patient posture, and/or patient activity level), and/or inputs that are patient-independent (e.g., time). Still further details are included in U.S. Pat. No. 8,355,797, incorporated herein by reference in its entirety.

In some embodiments, the signal generator 101 and/or signal delivery devices 110 can obtain power to generate the therapy signals from an external power source 103. For example, the external power source 103 can by-pass an implanted signal generator and generate a therapy signal directly at the signal delivery devices 110 (or via signal relay components). The external power source 103 can transmit power to the implanted signal generator 101 and/or directly to the signal delivery devices 110 using electromagnetic induction (e.g., RF signals). For example, the external power source 103 can include an external coil 104 that communicates with a corresponding internal coil (not shown) within the implantable signal generator 101, signal delivery devices 110, and/or a power relay component (not shown). The external power source 103 can be portable for ease of use.

In some embodiments, the signal generator 101 can obtain the power to generate therapy signals from an internal power source, in addition to or in lieu of the external power source 103. For example, the implanted signal generator 101 can include a non-rechargeable battery or a rechargeable battery to provide such power. When the internal power source includes a rechargeable battery, the external power source 103 can be used to recharge the battery. The external power source 103 can in turn be recharged from a suitable power source (e.g., conventional wall power).

During at least some procedures, an external stimulator or trial modulator 105 can be coupled to the signal delivery elements 110, e.g., during an initial procedure, prior to implanting the signal generator 101. For example, a practitioner (e.g., a physician and/or a company representative) can use the trial modulator 105 to vary the modulation parameters provided to the signal delivery elements 110 in real time, and select optimal or particularly efficacious parameters. These parameters can include the location from which the electrical signals are emitted, as well as the characteristics of the electrical signals provided to the signal delivery devices 110. In some embodiments, input is collected via the external stimulator or trial modulator 105 and can be used by the clinician to help determine what parameters to vary. In a typical process, the practitioner uses a cable assembly 120 to temporarily connect the trial modulator 105 to the signal delivery device 110. The practitioner can test the efficacy of the signal delivery devices 110 in an initial position. The practitioner can then disconnect the cable assembly 120 (e.g., at a connector 122), reposition the signal delivery devices 110, and reapply the electrical signals. This process can be performed iteratively until the practitioner obtains the desired position for the signal delivery devices 110. Optionally, the practitioner can move the partially implanted signal delivery devices 110 without disconnecting the cable assembly 120. Furthermore, in some embodiments, the iterative process of repositioning the signal delivery devices 110 and/or varying the therapy parameters may not be performed.

The signal generator 101, the lead extension 102, the trial modulator 105 and/or the connector 122 can each include a receiving element 109. Accordingly, the receiving elements 109 can be patient implantable elements, or the receiving elements 109 can be integral with an external patient treatment element, device or component (e.g., the trial modulator 105 and/or the connector 122). The receiving elements 109 can be configured to facilitate a simple coupling and decoupling procedure between the signal delivery devices 110, the lead extension 102, the pulse generator 101, the trial modulator 105 and/or the connector 122. The receiving elements 109 can be at least generally similar in structure and function to those described in U.S. Patent Application Publication No. 2011/0071593, which is incorporated by reference herein in its entirety.

After the signal delivery elements 110 are implanted, the patient 190 can receive therapy via signals generated by the trial modulator 105, generally for a limited period of time. During this time, the patient wears the cable assembly 120 and the trial modulator 105 outside the body. Assuming the trial therapy is effective or shows the promise of being effective, the practitioner then replaces the trial modulator 105 with the implanted signal generator 101, and programs the signal generator 101 with therapy programs selected based on the experience gained during the trial period. Optionally, the practitioner can also replace the signal delivery elements 110. In still further embodiments, the signal generator 101 can be implanted without first undergoing a trial period. Once the implantable signal generator 101 has been positioned within the patient 190, the therapy programs provided by the signal generator 101 can still be updated remotely via a wireless physician's programmer 117 (e.g., a physician's laptop, a physician's remote or remote device, etc.) and/or a wireless patient programmer 106 (e.g., a patient's laptop, patient's remote or remote device, etc.). Generally, the patient 190 has control over fewer parameters than does the practitioner. For example, the capability of the patient programmer 106 can be limited to starting and/or stopping the signal generator 101, and/or adjusting the signal amplitude within a present amplitude range. The patient programmer 106 can be configured to accept inputs corresponding to pain relief, motor functioning and/or other variables, such as medication use. Accordingly, more generally, embodiments of the present technology include receiving patient feedback, via a sensor, that is indicative of, or otherwise corresponds to, the patient's response to the signal. Feedback includes, but is not limited to, motor, sensory, and verbal feedback. In response to the patient feedback, one or more signal parameters can be adjusted, such as frequency, pulse width, amplitude, or delivery location.

FIG. 1B is a cross-sectional illustration of the spinal cord 191 and an adjacent vertebra 195 (based generally on information from Crossman and Neary, “Neuroanatomy,” 1995 (published by Churchill Livingstone)), along with multiple leads 111 (shown as leads 111a-111e) implanted at representative locations. For purposes of illustration, multiple leads 111 are shown in FIG. 1B implanted in a single patient. In addition, for purposes of illustration, the leads 111 are shown as elongated leads however, leads 111 can be paddle leads. In actual use, any given patient will likely receive fewer than all the leads 111 shown in FIG. 1B.

The spinal cord 191 is situated within a vertebral foramen 188, between a ventrally located ventral body 196 and a dorsally located transverse process 198 and spinous process 197. Arrows V and D identify the ventral and dorsal directions, respectively. The spinal cord 191 itself is located within the dura mater 199, which also surrounds portions of the nerves exiting the spinal cord 191, including the ventral roots 192, dorsal roots 193, and dorsal root ganglia 194. The dorsal roots 193 enter the spinal cord 191 at the dorsal root entry region 187, and communicate with dorsal horn neurons located at the dorsal horn 186. In some embodiments, the first and second leads 111a, 111b are positioned just off the spinal cord midline 189 (e.g., about 1 mm offset) in opposing lateral directions so that the two leads 111a, 111b are spaced apart from each other by about 2 mm, as discussed above. In some embodiments, a lead or pairs of leads can be positioned at other locations, e.g., toward the outer edge of the dorsal root entry portion 187 as shown by a third lead 111c, or at the dorsal root ganglia 194, as shown by a fourth lead 111d, or approximately at the spinal cord midline 189, as shown by a fifth lead 111e.

In some embodiments the devices and systems of the present technology include features other than those described herein. For example, one lead 111 to six leads 111 can be positioned generally end-to-end at or near the patient's midline M and span vertebral levels from about T4 to about T12. In some embodiments, two, three, or four leads 111 are positioned end-to-end at or near the patient's midline from T4 to T12. In some embodiments, the leads 111 and/or other signal delivery devices can have locations other than those expressly shown herein. For example, one or more signal delivery devices can be positioned at the dorsal side of the spinal cord 191. In addition, the devices and systems of the present technology can include more than one internal stimulator and/or more than one external stimulator that can be configured for wireless stimulation, such as by using electromagnetic waves.

Several aspects of the technology are embodied in computing devices, e.g., programmed/programmable pulse generators, controllers and/or other devices. The computing devices on/in which the described technology can be implemented can include one or more central processing units, memory, input devices (e.g., input ports), output devices (e.g., display devices), storage devices, and network devices (e.g., network interfaces). The memory and storage devices are computer-readable media that can store instructions that implement the technology. In some embodiments, the computer readable media are tangible media. In some embodiments, the data structures and message structures can be stored or transmitted via an intangible data transmission medium, such as a signal on a communications link. Various suitable communications links can be used, including but not limited to a local area network and/or a wide-area network.

FIG. 2 is a partially schematic illustration of a representative lead body 211 that can be used to apply modulation to a patient in accordance with any of the foregoing embodiments. In general, the lead body 211 includes a multitude of electrodes or contacts 220. When the lead body 211 has a circular cross-sectional shape, as shown in FIG. 2, the contacts 220 can have a generally ring-type shape and can be spaced apart axially along the length of the lead body 211. In a particular embodiment, the lead body 211 can include eight contacts 220, identified individually as first, second, third . . . eighth contacts 212, 222, 223 . . . 228. In general, one or more of the contacts 220 are used to provide signals, and another one or more of the contacts 220 provide a signal return path. Accordingly, the lead body 211 can be used to deliver monopolar modulation (e.g., if the return contact is spaced apart significantly from the delivery contact), or bipolar modulation (e.g., if the return contact is positioned close to the delivery contact and in particular, at the same target neural population as the delivery contact). In still further embodiments, the pulse generator 101 (FIG. 1A) can operate as a return contact for monopolar modulation.

### 4.0 Representative Therapy Signals

The present technology includes electrical therapy signals that can be delivered to a patient to treat one or more patient conditions (e.g., pain). For example, the system 100 described above can be programmed to generate and/or deliver any of the therapy signals described below. However, the therapy signals described herein can be generated and delivered using other neurostimulation systems, and are thus not limited to the systems explicitly described herein. Moreover, therapy regimens delivered in accordance with methods of the present technology can include any of the following therapy signals, including combinations thereof.

### 4.1 Existing High Frequency Therapy Signals

FIG. 3 is a partially schematic illustration of an existing high frequency neuromodulation signal 300. The signal 300 includes biphasic pulses 301 repeating in a continuous manner. Each individual pulse 301 includes an anodic pulse phase 302, a cathodic pulse phase 304, and an interphase interval 306 separating the anodic pulse phase 302 and the cathodic pulse phase 304. As described in greater detail below, the anodic pulse phase 302 and the cathodic pulse phase 304 are symmetrical (e.g., having generally equal pulse widths and generally equal and opposite amplitudes) such that individual pulses 301 are charge balanced. Individual pulses 301 are separated by an interpulse interval 308. Together, the pulse 301 and the interpulse interval 308 define a pulse period 310. The pulse period 310 repeats in cycles that define a frequency of the therapy signal 300.

As noted above, the pulse width of the anodic pulse phase 302 is generally equal to the pulse width of the cathodic pulse phase 304 for existing high frequency signals such as the illustrated signal 300. The anodic pulse phase 302 and the cathodic pulse phase 304 can each have a pulse width in a pulse width range of from about 1 microsecond or less and about 416 microseconds. In further embodiments, the anodic pulse phase 302 and the cathodic pulse phase 304 can each have a pulse width in a pulse width range of from about 10 microseconds to about 333 microseconds, or from about 25 microseconds to about 166 microseconds, or from about 33 microseconds to about 100 microseconds, or from about 50 microseconds to about 166 microseconds. The amplitude of the anodic pulse phase 302 and the cathodic pulse phase 304 also generally have equal values of opposite polarity. In some embodiments, the anodic pulse phase 302 and the cathodic pulse phase 304 can each have an amplitude in an amplitude range of from about 0.1 mA to about 20 mA. In further embodiments, the anodic pulse phase 302 and the cathodic pulse phase 304 can each have an amplitude in an amplitude range of from about 0.5 mA to about 10 mA, or about 0.5 mA to about 4 mA, or about 0.5 mA to about 2.5 mA. The amplitude of the therapy signal 300 can optionally be ramped up and/or down. The frequency of the therapy signal 300 can be in a frequency range of from about 1.2 kHz to about 100 kHz. In further embodiments, the therapy signal 300 can have a frequency in a frequency range of from about 1.2 kHz to about 50 KHz, from about 1.2 kHz to about 25 kHz, from about 3 kHz to about 15 kHz, or from about 5 kHz to about 15 kHz. For example, in some embodiments the therapy signal 300 has a frequency of about 5 kHz, about 10 KHz, about 15 kHz, about 20 kHz, about 25 kHz, about 50 kHz, or about 100 kHz. Additional details of symmetric therapy signals are described in U.S. Pat. No. 8,712,533, which is incorporated by reference herein in its entirety.

4.2 Therapy Signals with Asymmetric Bi-Phasic Pulses

FIGS. 4A-4C are partially schematic illustrations of representative asymmetric electrical therapy signals 400a-400c (collectively referred to as the “signals 400”) used to deliver therapy in accordance with embodiments of the present technology. The signals 400 each include asymmetric bi-phasic pulses 401a-c (collectively referred to as the “asymmetric bi-phasic pulses 401,” or simply “pulses 401”), with each asymmetric bi-phasic pulse 401 including an anodic pulse phase 402a-c (collectively referred to as the “anodic pulse phase 402”), a cathodic pulse phase 404a-c (collectively referred to as the “cathodic pulse phase 404”), and an interphase interval 406a-c (collectively referred to as the “interphase interval 406”) separating the anodic pulse phase 402 and the cathodic pulse phase 404. Although the signals 400 are shown in FIGS. 4A-4C with the anodic pulse phase 402 as the “leading phase” of the pulses 401, in other embodiments the cathodic pulse phase 404 can be the “leading phase” of the pulses 401, followed by the anodic pulse phase 402. Unlike the therapy signal 300 described with reference to FIG. 3, the anodic pulse phases 402 and the cathodic pulse phase 404 are asymmetric (e.g., they have unequal pulse widths and amplitudes). As described in greater detail below, the individual pulses 401 are nevertheless charge balanced if the total charge delivered during the anodic pulse phase 402 and the cathodic pulse phase 404 are equal and opposite. Individual bi-phasic pulses 401 are separated by an interpulse interval 408a-c (collectively referred to as the “interpulse interval 408”). Together, the pulses 401 and the interpulse intervals 408 define a pulse period 410a-c (collectively referred to as the “pulse period 410”). The pulse period 410 repeats in cycles that define a frequency of the signal 400.

As noted above, the asymmetric bi-phasic pulses 401 have anodic pulse phases 402 and cathodic pulse phases 404 having unequal pulse widths and amplitudes. In some embodiments, the cathodic pulse phases 404 have greater pulse widths than the corresponding anodic pulse phases 402. For example, the ratio between the cathodic pulse phase 404 pulse width and the anodic pulse phase 402 pulse width can be between about 1.2:1 and about 8:1, or between about 2:1 and about 6:1, or between about 2:1 and about 5:1, or between about 2:1 and about 4:1. In particular examples, the ratio can be about 2:1, about 3:1, about 4:1, about 5:1, about 6:1, about 7:1 or about 8:1. To maintain an equal and offset charge during a single bi-phase pulse 401, the cathodic pulse phases 404 can have a smaller amplitude than the corresponding anodic pulse phase 402. For example, the ratio between the cathodic pulse phase 404 amplitude absolute value and the anodic pulse phase 402 amplitude absolute value can be between about 1:1.2 and about 1:8, or between about 1:2 and about 1:6, or between about 1:2 and about 1:5, or between about 1:2 and about 1:4. In particular examples, the ratio can be about 1:2, about 1:3, about 1:4, about 1:5, about 1:6, about 1:7, or about 1:8. In some embodiments, the ratio of the cathodic pulse phase amplitude to the anodic pulse phase amplitude is the inverse of the ratio of the cathodic pulse phase pulse width to the anodic pulse phase pulse width to ensure an equal and opposite charge is delivered during each pulse 401.

In other embodiments, the anodic pulse phases 402 can have greater pulse widths than the corresponding cathodic pulse phases 404. For example, the ratio between the anodic pulse phase 402 pulse width and the cathodic pulse phase 404 pulse width can be between about 1.2:1 and about 8:1, or between about 2:1 and about 6:1, or between about 2:1 and about 5:1, or between about 2:1 and about 4:1. In such embodiments, the anodic pulse phases 402 can have a smaller amplitude than the corresponding cathodic pulse phases 404. For example, the ratio between the anodic pulse phase 402 amplitude absolute value and the cathodic pulse phase 404 amplitude absolute value can be between about 1:1.2 and about 1:8, or between about 1:2 and about 1:6, or between about 1:2 and about 1:5, or between about 1:2 and about 1:4.

In some embodiments, one pulse phase (e.g., the anodic pulse phase 402 or the cathodic pulse phase 404) provides a therapeutic effect and the other pulse phase (e.g., the other of the anodic pulse phase 402 or the cathodic pulse phase 404) provides charge balancing. Accordingly, in some embodiments the signals 400 can be described as having a therapy pulse phase and a charge balancing pulse phase. In some embodiments, the combination of the two pulse phases (e.g., the anodic pulse phase 402 and the cathodic pulse phase 404) provide the therapeutic effect.

In embodiments in which the cathodic pulse phase 404 pulse width is greater than the anodic pulse phase 402 pulse width, the anodic pulse phase 402 can have a pulse width in a pulse width range of from about 1 microsecond or less and about 416 microseconds, and the cathodic pulse phase 404 can have a pulse width in a pulse width range of from about 1.2 microseconds or less to about 740 microseconds. In further embodiments, the anodic pulse phase 402 can have a pulse width in a pulse width range of from about 10 microseconds to about 333 microseconds, or from about 25 microseconds to about 166 microseconds, or from about 33 microseconds to about 100 microseconds, or from about 50 microseconds to about 166 microseconds, and the cathodic pulse phase 404 can have a pulse width in a pulse width range of from about 12 microseconds and about 740 microseconds, or from about 30 microseconds to about 333 microseconds, or from about 40 microseconds to about 200 microseconds, or from about 60 microseconds to about 333 microseconds. In embodiments in which the anodic pulse phase 402 pulse width is greater than the cathodic pulse phase 404 pulse width, the foregoing ranges of pulse widths are reversed (e.g., the cathodic pulse phase can have a pulse width in a pulse width range of from about 1 microsecond or less to about 416 microseconds, and the anodic pulse phase can have a pulse width in a pulse width range of from about 1.2 microseconds or less to about 700 microseconds). The foregoing pulse widths are a function of the frequency of the signals 400 shown in FIGS. 4A-4C, the ratio of the cathodic pulse phase 402 pulse width to the anodic pulse phase 404 pulse width, and any interpulse or interphase interval.

In embodiments for which the anodic pulse phase 402 amplitude is greater than the cathodic pulse phase 404 amplitude, the anodic pulse phase 402 can have an amplitude in an amplitude range of from about 0.1 mA to about 20 mA, and the cathodic pulse phase 404 can have an amplitude in an amplitude range of from about 0.02 mA to about 16 mA. In further embodiments, the anodic pulse phase 402 can have an amplitude in an amplitude range of from about 0.5 mA to about 10 mA, or about 0.5 mA to about 4 mA, or about 0.5 mA to about 2.5 mA, and the cathodic pulse phase 404 can have an amplitude in an amplitude range of from about 0.1 mA to about 8 mA, or about 0.1 mA to about 3.2 mA, or about 0.1 mA to about 2 mA. In embodiments in which the anodic pulse phase 402 amplitude is less than the cathodic pulse phase 404 amplitude, the foregoing ranges of amplitudes are reversed (e.g., the cathodic pulse phase 404 can have an amplitude in an amplitude range of from about 0.1 mA to about 20 mA, and the anodic pulse phase 402 can have an amplitude in an amplitude range of from about 0.2 mA to about 16 mA).

The signals 400 can have a frequency in a frequency range of from about 1.2 KHz to about 500 KHz or even higher at narrow pulse widths. In some embodiments, the signals 400 have a frequency of from about 1.2 kHz to about 100 kHz, from about 1.2 kHz to about 50 kHz, from about 1.2 kHz to about 25 kHz, from about 3 kHz to about 15 kHz, or from about 5 kHz to about 15 kHz. For example, in some embodiments the signal 400 has a frequency of about 1.2 kHz, about 1.5 kHz, about 5 kHz, about 10 KHz, about 15 kHz, about 20 kHz, about 25 kHz, about 50 kHz, or about 100 KHz.

FIG. 4A illustrates a representative asymmetric electrical signal 400a comprising asymmetric bi-phasic pulses 401a having a cathodic pulse phase 404a with a pulse width that is approximately twice as long as the anodic pulse phase 402a pulse width (e.g., the ratio between the cathodic pulse phase 404a pulse width and the anodic pulse phase 402a pulse width is 2:1). To maintain an equal and offset charge during the bi-phase pulse 401a, the cathodic pulse phase 404a has an amplitude that has an absolute value that is approximately one-half of the absolute value of the amplitude of the anodic pulse phase 402a (e.g., the ratio between the absolute value of the cathodic pulse phase 404a amplitude and the anodic pulse phase 402a amplitude is 1:2). Accordingly, the total charge delivered during the anodic pulse phase 402a and the cathodic pulse phase 404a is equal and opposite, and the asymmetric bi-phasic pulse 401a is charge balanced. In one example, the anodic pulse phase 402a has a pulse width of about 20 microseconds, the cathodic pulse phase 404a has a pulse width of about 40 microseconds, and the signal 400a has a frequency of about 5 KHz.

FIG. 4B illustrates a representative asymmetric electrical signal 400b comprising asymmetric bi-phasic pulses 401b having a cathodic pulse phase 404b with a pulse width that is approximately three times as long as the anodic pulse phase 402b pulse width (e.g., the ratio between the cathodic pulse phase 404b pulse width and the anodic pulse phase 402b pulse width is 3:1). To maintain an equal and offset charge during the bi-phase pulse 401b, the cathodic pulse phase 404b has an amplitude that has an absolute value that is approximately one-third of the absolute value of the amplitude of the anodic pulse phase 402b (e.g., the ratio between the absolute value of the cathodic pulse phase 402b amplitude and the anodic pulse phase 402b amplitude is 1:3). Accordingly, the total charge delivered during the anodic pulse phase 402b and the cathodic pulse phase 404b is equal and opposite, and the asymmetric bi-phasic pulse 401b is charge balanced. In one example, the anodic pulse phase 402b has a pulse width of about 20 microseconds, the cathodic pulse phase 404b has a pulse width of about 60 microseconds, and the signal 400a has a frequency of about 5 kHz.

FIG. 4C illustrates a representative asymmetric electrical signal 400c comprising asymmetric bi-phasic pulses 401c having a cathodic pulse phase 404c with a pulse width that is approximately six times as long as the anodic pulse phase 402c pulse width (e.g., the ratio between the cathodic pulse phase 404c pulse width and the anodic pulse phase 402c pulse width is 6:1). To maintain an equal and offset charge during the bi-phase pulse 401c, the cathodic pulse phase 404c has an amplitude that has an absolute value that is approximately one-sixth of the absolute value of the amplitude of the anodic pulse phase 402c (e.g., the ratio between the absolute value of the cathodic pulse phase 402c amplitude and the anodic pulse phase 402c amplitude is 1:6). Accordingly, the total charge delivered during the anodic pulse phase 402c and the cathodic pulse phase 404c is equal and opposite, and the asymmetric bi-phasic pulse 401c is charge balanced. In one example, the anodic pulse phase 402c has a pulse width of about 20 microseconds, the cathodic pulse phase 404c has a pulse width of about 120 microseconds, and the signal 400a has a frequency of about 5 KHz.

Without being bound by any particular theory, the use of asymmetric pulses during neurostimulation therapy may modulate a target population of neurons in a different manner than the use of existing symmetrical pulses. For example, asymmetric pulses may preferentially modulate a first set of neurons (e.g., adapting neurons, excitatory neurons, etc.) within a target set of neurons, whereas existing symmetrical pulses may preferentially modulate a second set of neurons (e.g., nonadapting neurons, inhibitory neurons, etc.) within the target set of neurons. In some embodiments, the asymmetric pulses may modulate both the first set of neurons and the second set of neurons, but in a different manner than and/or to a different degree than (e.g., greater than, less than, etc.) existing symmetrical pulses. Therefore, in some embodiments, signals having asymmetric pulses can be delivered to achieve a first physiological effect or result, and signals having symmetric pulses can be delivered to achieve a second physiological effect or result. The first and second physiological effects can be complementary, synergistic, or antagonistic. Additional details regarding potential effects of, and mechanisms of action for, asymmetric pulses are described in Section 6.0 below.

4.3 Therapy Signals with Relatively Long Inter-Phase Intervals

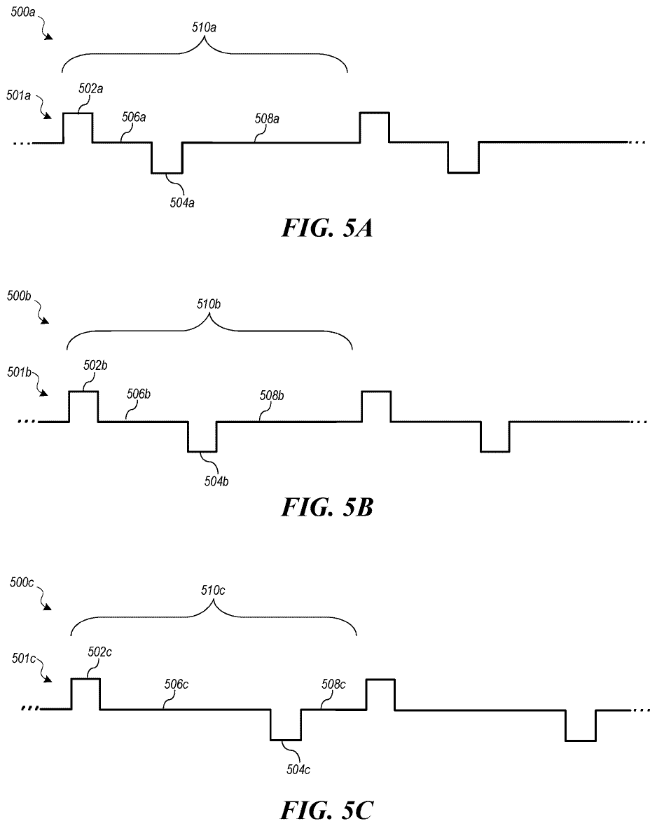

FIGS. 5A-5C are partially schematic illustrations of representative electrical therapy signals 500a-500c (collectively referred to as the “signals 500”) used to deliver therapy in accordance with embodiments of the present technology. The signals 500 each include bi-phasic pulses 501a-c (collectively referred to as the “bi-phasic pulses 501,” or simply “pulses 501”), with each bi-phasic pulse 501 including an anodic pulse phase 502a-c (collectively referred to as the “anodic pulse phase 502”), a cathodic pulse phase 504a-c (collectively referred to as the “cathodic pulse phase 504”), and an interphase interval 506a-c (collectively referred to as the “interphase interval 506”) separating the anodic pulse phase 502 and the cathodic pulse phase 504. Although the therapy signals 500 are shown in FIGS. 5A-5C with the anodic pulse phase 502 as the “leading phase” of the pulses 501, in other embodiments the cathodic pulse phase 504 can be the “leading phase” of the pulses 501, followed by the anodic pulse phase 502. Moreover, although described as having anodic pulse phases and cathodic pulse phases, the signals 500 can also be described as having a therapy pulse phase (e.g., shown in FIGS. 5A-5C as the anodic pulse phase 502) followed by a charge balancing pulse phase (e.g., shown in FIGS. 5A-5C as the cathodic pulse phase 504). The interphase interval 506 is the duration between the leading pulse phase (e.g., the therapy pulse phase) and the following pulse phase (e.g., the charge balancing pulse phase). Individual bi-phasic pulses 501 are separated by an interpulse interval 508a-c (collectively referred to as the “interpulse interval 508”). Together, the pulses 501 and the interpulse intervals 508 define a pulse period 510a-c (collectively referred to as the “pulse period 510”). The pulse period 510 repeats in cycles that define a frequency of the signal 500.

Unlike the therapy signals 300 and 400 described with reference to FIGS. 3-4C, the signals 500 shown in FIGS. 5A-5C have an interphase interval 506 that is greater than the pulse width of the leading pulse phase, which in the embodiment illustrated in FIGS. 5A-5C is the anodic pulse phase 502. Accordingly, the term “relatively long interphase interval” is used herein to refer to an interphase interval having a duration greater than the pulse width of the leading pulse phase. A ratio between the duration of the interphase interval 506 and the pulse width of the leading pulse phase can be between about 1.2:1 and about 8:1, or between about 2:1 and about 6:1, or between about 2:1 and about 5:1, or between about 2:1 and about 4:1. In some embodiments, the ratio is about 2:1, about 3:1, about 4:1, about 5:1, about 6:1, about 7:1, or about 8:1. In some embodiments, the duration of the relatively long interphase interval is between about 24 microseconds and about 160 microseconds, or between about 30 microseconds and about 120 microseconds, or between about 40 microseconds and 80 microseconds, or about 60 microseconds. In some embodiments, an increase in the interphase interval 506 is accompanied by a corresponding decrease in the interpulse interval 508, such that the duration of the pulse period 610, and therefore the frequency, remains unchanged.

While the interphase interval 506 is proportionally larger than the interphase interval 306 of the signal 300 shown in FIG. 3, the pulse width, amplitudes, and frequencies of the signal 500 can be generally the same as those described with respect to the signals 300 and/or 400. For example, in some embodiments the pulse width of the anodic pulse phase 502 is generally equal to the pulse width of the cathodic pulse phase 504, although in other embodiments the anodic pulse phase 502 pulse width is not equal to the cathodic pulse phase 504 pulse width. In some embodiments, the anodic pulse phase 502 and the cathodic pulse phase 504 can each have a pulse width in a pulse width range of from about 1 microsecond or less and about 416 microseconds. In further embodiments, the anodic pulse phase 502 and the cathodic pulse phase 504 can each have a pulse width in a pulse width range of from about 10 microseconds to about 333 microseconds, or from about 25 microseconds to about 166 microseconds, or from about 33 microseconds to about 100 microseconds, or from about 50 microseconds to about 166 microseconds.

The amplitude of the anodic pulse phase 302 and the cathodic pulse phase 304 also generally have equal values of opposite polarity. In some embodiments, the anodic pulse phase 502 and the cathodic pulse phase 504 can each have an amplitude in an amplitude range of from about 0.1 mA to about 20 mA. In some embodiments, the anodic pulse phase 502 and the cathodic pulse phase 504 can each have an amplitude in an amplitude range of from about 0.5 mA to about 10 mA, or about 0.5 mA to about 4 mA, or about 0.5 mA to about 2.5 mA. The amplitude of the signals 500 can optionally be ramped up and/or down. The frequency of the signals 500 can be in a frequency range of from about 1.2 kHz to about 500 KHz. In some embodiments, the therapy signals 500 can have a frequency in a frequency range of from about 1.2 kHz to about 100 kHz, from about 1.2 kHz to about 50 kHz, from about 1.2 kHz to about 25 kHz, from about 3 kHz to about 15 kHz, or from about 5 kHz to about 15 kHz. For example, in some embodiments the signals 500 have a frequency of about 1.2 kHz, about 1.5 kHz, about 5 kHz, about 10 kHz, about 15 kHz, about 20 kHz, about 25 kHz, about 50 kHz, or about 100 KHz.

FIG. 5A illustrates a representative electrical signal 500a having an interphase interval 506a that is twice as long as the pulse width of the anodic pulse phase 502a (e.g., the ratio between the duration of the interphase interval 506a and the anodic pulse phase 502a pulse width is 2:1). In one example, the anodic pulse phase 502a has a pulse width of about 20 microseconds, the interphase interval 506b has a duration of about 40 microseconds, and the signal 500a has a frequency of about 5 KHz.

FIG. 5B illustrates a representative electrical signal 500b having an interphase interval 506b that is three times as long as the pulse width of the anodic pulse phase 502b (e.g., the ratio between the duration of the interphase interval 506b and the anodic pulse phase 502b pulse width is 3:1). In one example, the anodic pulse phase 502b has a pulse width of about 20 microseconds, the interphase interval 506b has a duration of about 60 microseconds, and the signal 500b has a frequency of about 5 KHz.

FIG. 5C illustrates a representative electrical signal 500c having an interphase interval 506c that is six times as long as the pulse width of the anodic pulse phase 502c (e.g., the ratio between the duration of the interphase interval 506c and the anodic pulse phase 502c pulse width is 6:1). In one example, the anodic pulse phase 502c has a pulse width of about 20 microseconds, the interphase interval 506c has a duration of about 120 microseconds, and the signal 500a has a frequency of about 5 KHz.

Without being bound by any particular theory, the use of electrical signals having relatively long interphase intervals may modulate a target population of neurons in a different manner than existing electrical signals. For example, the signals 500 may preferentially modulate a first set of neurons (e.g., adapting neurons, excitatory neurons, etc.) within a target set of neurons, whereas existing signals (e.g., those in which the interphase interval is equal to or less than the pulse width of the anodic pulse phase) may preferentially modulate a second set of neurons (e.g., nonadapting neurons, inhibitory neurons, etc.) within the target set of neurons. In some embodiments, the signals 500 may modulate both the first set of neurons and the second set of neurons, but in a different manner and/or to a different degree than (e.g., greater than, less than, etc.) existing signals. Therefore, in some embodiments, signals having relatively long interphase intervals can be delivered to achieve a first physiological effect or result, and signals having existing interphase intervals can be delivered to achieve a second physiological effect or result. The first and second physiological effects can be complementary, synergistic, or antagonistic. Additional details regarding potential effects of, and mechanisms of action for, biphasic pulses having relatively long interphase intervals are described in Section 6.0 below.

### 5.0 Representative Patterns of Administration

In some embodiments, electrical signals in accordance with the present technology can be administered via more than two electrodes or contacts, with each pulse of the signal delivered via a pair of contacts that is different than the pair of contacts used to deliver the immediately preceding pulse. FIG. 6A illustrates an example of such a pattern of administration, which can also be referred to herein as a “juggling” pattern. More specifically, FIG. 6A illustrates an electrical therapy signal 600 having mono-phasic pulses (shown as a first mono-phasic pulse 601a, a second mono-phasic pulse 601b, and a third mono-phasic pulse 601c).

FIG. 6A further illustrates a signal delivery element or lead 620 having a first contact or electrode 621, a second contact or electrode 622, and a third contact or electrode 623. The signal delivery element 620 is shown three times in FIG. 6A to illustrate the pattern of contact(s) used to deliver the pulses. The first pulse 601a is delivered via the first contact 621 and the second contact 622 (e.g., electrical current flows out of the first contact 621 and into the second contact 622). The second pulse 601b is subsequently delivered via the second contact 622 and the third contact 623 (e.g., electrical current flows out of the second contact 622 and into the third contact 623). The third pulse 601c is subsequently delivered via the third contact 623 and the first contact 621 (e.g., electrical current flows out of the third contact 623 and into the first contact 622). This pattern of administration can be repeated with subsequent pulses of the signal 600, such that no two adjacent pulses are delivered via the same pair of contacts. Although shown with three contacts, the signal 600 can be administered according to the juggling pattern described above with any number of contacts greater than two, such as three, four, five, six, seven, eight, nine, ten, or more contacts. The pairing of electrodes can also be changed for any given pulse. For example, electrical current could flow from the first contact 621 to the third contact 623 during a first pulse, from the third contact 623 to the second contact 622 during a second pulse, and from the second contact 622 to the first contact 621 during a first pulse.

Despite the signal 600 being mono-phasic, the contacts 621-623 do not build up significant charge because each contact delivers and receives the same amount of current. FIG. 6B is a schematic representation of the charge at each contact 621-623 during administration of the pulses 601a-601c. For the first pulse 601a, the first contact 621 has a positive charge change, the second contact 622 has a negative charge change, and the third contact has no charge change. For the second pulse 601b, the first contact 621 has no charge change, the second contact 622 has a positive charge change, and the third contact 623 has a negative charge change. For the third pulse 601c, the first contact has a negative charge change, the second contact 622 has no charge change, and the third contact has a positive charge change. Therefore, the first contact 621 experiences a positive charge during the first pulse 601a that is balanced by the negative charge during the third pulse 601c, the second contact 622 experiences a negative charge during the first pulse 601a that is balanced by the positive charge during the second pulse 601b, and the third contact 623 experiences a negative charge during the second pulse 601b that is balanced by the positive charge during the third pulse 601c. Accordingly, for delivery patterns using three contacts such as those illustrated, combining three mono-phasic pulses forms a set of pulses that results in each electrode being charge balanced. The number of mono-phasic pulses forming the charge balanced pulse set depends on the number of electrodes used. For example, if four electrodes are used then four monophasic pulses form the charge balanced set of pulses, if five electrodes are used then five monophasic pulses form the charge balanced set of pulses, and so on.

Signals having asymmetric pulses can also be delivered according to the juggling pattern while maintaining charge-balancing at the contacts 621-623, provided the total charge at each electrode is offset. For example, FIG. 6C illustrates a monophasic signal 650 having a plurality of pulses, each having a different amplitude and pulse width (e.g., shown as a first monophasic pulse 651a, a second monophasic pulse 651b, and a third monophasic pulse 651c). Of note, the amplitude and pulse widths of the pulses 651a-c are inversely proportional such that the total charge delivered by each pulse is the same or at least substantially the same. FIG. 6C also shows the charge at each contact 621-623 during administration of the pulses 651a-c. Because the total charge delivered by each pulse 651a-c is the same, the contacts 621-623 remain substantially charge balanced after delivery of the three pulses 651a-651c.

The foregoing pattern is different than both existing bipolar and tripolar patterns of administration (referred to herein as “existing administration patterns”), which are used to administer conventional bi-phasic pulses. In existing bipolar administrations, a single pair of contacts is used to deliver the signal, with a first contact delivering the anodic pulse and a second contact delivering the cathodic pulse. In existing tripolar patterns of administration, three contacts are used to deliver the signal, with a first contact delivering the anodic pulse and second and third contacts positioned on opposing sides of the first contact delivering the anodic pulses (or vice versa). In both existing bipolar and tripolar patters, the contacts generally remain as either an anode or a cathode, and do not switch between phases. In contrast, when using the juggling pattern described herein, a single contact can act as an anode for a first pulse, a cathode for a second pulse, and be neutral for a third pulse. This increases the area of the current loop extending between the contacts during administration of the electrical signal, and thus may increase the biological area that is affected by (e.g., the number of neurons recruited by) the electrical signals.

Without being bound by any particular theory, administering electrical signals via a juggling pattern may modulate a target population of neurons in a different manner than administering the same electrical signal via existing patterns. For example, administering a signal using the juggling pattern may preferentially modulate a first set of neurons (e.g., adapting neurons, excitatory neurons, etc.) within a target set of neurons, whereas administering the signal using an existing pattern may preferentially modulate a second set of neurons (e.g., nonadapting neurons, inhibitory neurons, etc.) within the target set of neurons. In some embodiments, administering a signal using the juggling pattern may modulate both the first set of neurons and the second set of neurons, but in a different manner than and/or to a different degree than administering a signal using an existing pattern of administration. In some embodiments, administering a signal using the juggling patterns may modulate a larger target area (thus modulating more neurons) than the same signal administered via an existing pattern. Therefore, in some embodiments, signals can be delivered using the juggling pattern to achieve a first physiological effect or result, and signals can be delivered using the existing pattern to achieve a second physiological effect or result. The first and second physiological effects can be complementary, synergistic, or antagonistic. Additional details regarding potential effects of, and mechanisms of action for, the juggling pattern of administration are described in Section 6.0 below.

### 6.0 Selectivity of Modified High Frequency Neuromodulation Signals

As described above, the modified high frequency neuromodulation signals (e.g., the asymmetric signals described with respect to FIGS. 4A-4C, the signals having relatively long interphase intervals described with respect to FIGS. 5A-5C, and signals delivered in accordance with a juggling pattern as described with respect to FIGS. 6A-6C) can modulate a target population of neurons in a different manner than existing high frequency neuromodulation signals administered via an existing pattern. FIG. 7 is a graph showing the firing rate of excitatory neurons and inhibitory neurons in response to select high frequency neuromodulation signals. Line 701 reflects the relative firing rate of excitatory versus inhibitory neurons for an existing 10 KHz electrical signal administered at various percentages of a motor threshold. Line 702 reflects the relative firing rate of excitatory versus inhibitory neurons for an existing 50 KHz electrical signal administered at various percentages of a motor threshold. Line 703 reflects the relative firing rate of excitatory versus inhibitory neurons for an electrical signal administered according to a “juggling” pattern and at various percentages of a motor threshold.

Without intending to be bound by any particular theory, and as shown by lines 701 and 702, existing high frequency electrical signals can preferentially activate inhibitory neurons relative to excitatory neurons regardless of the intensity of the signal. For example, a 10 KHz electrical signal administered at 30% of motor threshold induces a 10 Hz firing rate in inhibitory neurons but only a 2 Hz firing rate in excitatory neurons, while the same 10 kHz electrical signal administered at 60% of motor threshold induces a 27 Hz firing rate in excitatory neurons but only a 6 Hz firing rate in excitatory neurons. Likewise, a 50 KHz electrical signal administered at 30% of motor threshold induces about a 23 Hz firing rate in inhibitory neurons but only about a 4 Hz firing rate in excitatory neurons.

In contrast, modified high frequency neuromodulation signals can modulate a target population of neurons in a different manner. As shown by line 703, administering an electrical signal according to a juggling pattern can have a greater influence on excitatory neurons than inhibitory neurons, particularly as the intensity of the administered signal increases toward motor threshold. For example, while administering the signal at 10% motor threshold appears to preferentially activate inhibitory neurons, administering the signal at 30% motor threshold and above appears to preferentially activate excitatory neurons.

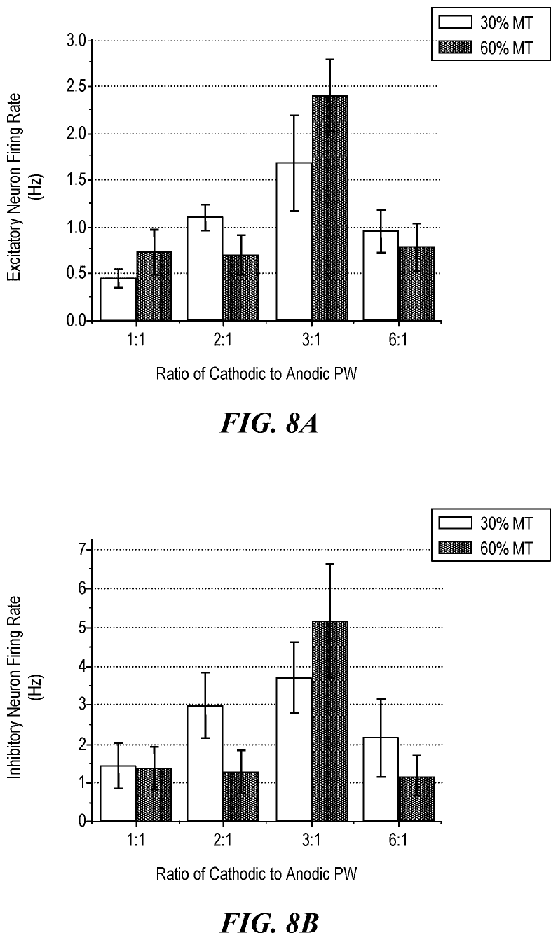

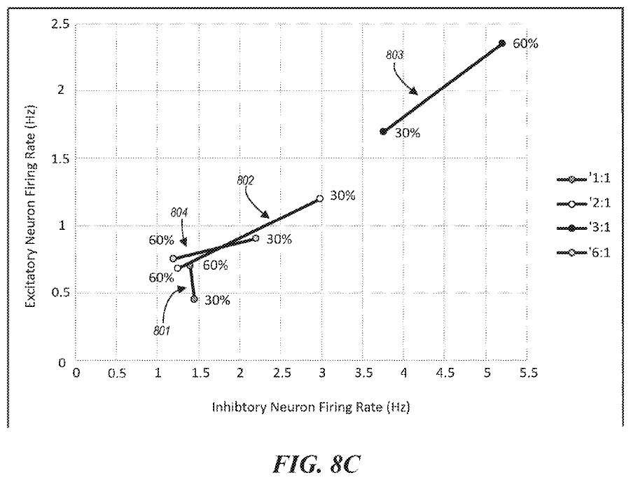

FIGS. 8A-8C are graphs further demonstrating the effects of modified high frequency neuromodulation signals having asymmetric bi-phasic pulses (e.g., the signals 400 described with respect to FIGS. 4A-4C). More specifically, FIG. 8A illustrates the firing rate of excitatory neurons in response to an existing 5 kHz electrical signal having symmetric bi-phasic pulses with a cathodic pulse phase pulse width to anodic pulse phase pulse width ratio of 1:1, and several modified 5 kHz neuromodulation signals having asymmetric bi-phasic pulses with cathodic pulse phase pulse width to anodic pulse phase pulse width ratios of 2:1, 3:1, and 6:1. Data is shown for signals administered at both 30% motor threshold and 60% motor threshold. FIG. 8B illustrates the same, but for inhibitory neurons. FIG. 8C is a line graph comparing the firing rate of excitatory neurons versus the firing rate of inhibitory neurons. In particular, line 801 reflects the relative firing rate of excitatory versus inhibitory neurons for the existing 5 kHz symmetric signal having a 1:1 ratio at various percentages of motor threshold, and line 802 reflects the relative firing rate of excitatory versus inhibitory neurons for the 5 kHz asymmetric signal having a 2:1 ratio at various percentages of motor threshold. Additionally, line 803 reflects the relative firing rate of excitatory versus inhibitory neurons for the 5 kHz asymmetric signal having a 3:1 ratio at various percentages of motor threshold, and line 804 reflects the relative firing rate of excitatory versus inhibitory neurons for the 5 kHz asymmetric signal having a 6:1 ratio at various percentages of motor threshold.

As shown in FIGS. 8A-8C, the 5 kHz signals with asymmetric bi-phasic pulses did not preferentially activate excitatory neurons but nevertheless generally activated excitatory neurons at a greater rate than did the existing symmetric 5 kHz signal. Moreover, the 5 kHz asymmetric signal with a 3:1 ratio between the cathodic pulse phase pulse width and the anodic pulse phase pulse width generally activated both inhibitory neurons and excitatory neurons at the highest rate compared to the other signals. Without intending to be bound by any particular theory, a ratio of 3:1 may induce the strongest activation because, at such ratio, the cathodic pulse phase has the least impact on neuronal response to the anodic pulse phase. At higher ratios, the cathodic pulse phase may impact neuronal response to the anodic pulse phase of the next pulse. At lower ratios, the cathodic pulse phase may impact neuronal response to the paired anodic pulse phase.

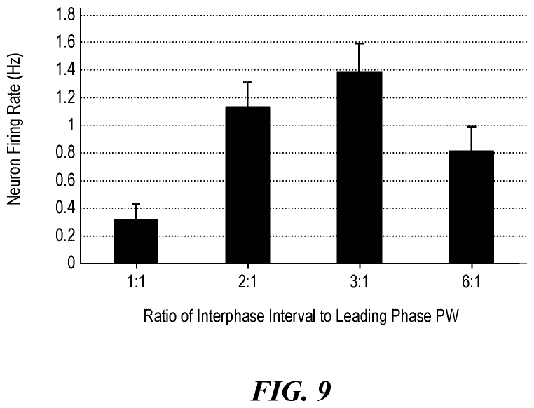

FIG. 9 is a graph further demonstrating the selective modulation effects of modified high frequency neuromodulation signals having relatively long interphase intervals (e.g., the signals 500 described with respect to FIGS. 5A-5C). More specifically, FIG. 9 illustrates the firing rate of neurons in response to an existing 5 kHz electrical signal having bi-phasic pulses with an interphase interval to leading phase pulse width ratio of 1:1 (e.g., pulses having a leading anodic pulse phase pulse width of 20 microseconds and an interphase interval of 20 microseconds). FIG. 9 also illustrates several modified 5 kHz neuromodulation signals having relatively long interphase intervals, and having interphase interval to leading phase pulse width ratios of 2:1, 3:1, and 6:1 (e.g., pulses having a leading anodic pulse phase pulse width of 20 microseconds and an interphase interval of 40 microseconds, 60 microseconds, and 120 microseconds, respectively). The data shown in FIG. 9 was obtained invitro by increasing the amplitude of the various signals until a neuronal response was detected.

The modified 5 kHz signals with relatively long interphase intervals generally activated neurons at a greater rate than did the existing 5 kHz signal. Moreover, the modified 5 kHz signal with a 3:1 ratio between the interphase interval and the leading phase pulse width generally activated neurons at the highest rate compared to the other signals. Without intending to be bound by any particular theory, a ratio of 3:1 may induce the strongest neuronal response because, at such a ratio, the second pulse phase of the pulse (e.g., the cathodic pulse phase) may have less impact on neuron response to the leading pulse phase of the pulse (e.g., the anodic pulse phase). At high ratios, the second pulse phase of the pulse may impact neuronal response to the leading pulse phase of the next pulse. At lower ratios, the second pulse phase of the pulse may impact neuronal response to the paired leading pulse phase.

In some embodiments, and without being bound by theory, the different activation patterns induced by existing (e.g., symmetric bi-phasic signals having relatively short interphase intervals and delivered using standard patterns) versus modified high frequency neuromodulation signals can also be described in terms of adapting versus non-adapting neurons. For example, existing high frequency signals can preferentially activate non-adapting neurons, while modified high frequency signals can preferentially activate adapting neurons (or at least activate a different fraction of adapting versus non-adapting neurons).

In some patients, the different activation patterns induced by existing versus modified high frequency neuromodulation signals may be explained based on the reach of the signals. Without being bound by theory, modified high frequency signals may activate neurons in a larger and/or different area than existing high frequency signals. For example, the modified high frequency signals may activate excitatory neurons that project to and therefore activate inhibitory interneurons that are outside the reach of existing high frequency signals. By activating neurons in a larger and/or different area, the modified high frequency neuromodulation signals increase the “reach” of the signal and thus result in increased activation of both excitatory and inhibitory neurons.

### 7.0 Combination Therapy

Any of the neuromodulation signals and administration patterns described herein can be used in combination. For example, the neuromodulation systems can be programmed with instructions for delivering existing high frequency signals, modified high frequency signals having asymmetric bi-phasic pulses, and/or modified high frequency signals having relatively long interphase intervals. Combination therapy can include delivering two or more different waveforms temporally (e.g., delivering a modified high frequency signal having asymmetric bi-phasic pulses or relatively long interphase intervals for a first period of time, and delivering existing high frequency signals for a second period of time after the first period of time using). In some embodiments, the first period of time may at least partially overlap with the second period of time. In other embodiments, the first period of time does not overlap with the second period of time. Combination therapy can also include delivering two or more different waveforms spatially (e.g., delivering a modified high frequency signal having asymmetric bi-phasic pulses or relatively long interphase intervals using a first set of electrodes, and delivering existing high frequency signals using a second set of electrodes). Combination therapy can also include delivering two or more different waveforms temporo-spatially (e.g., delivering a modified high frequency signal having asymmetric bi-phasic pulses or relatively long interphase intervals for a first period of time using a first set of electrodes, and delivering existing high frequency signals for a second period of time after the first period of time using a second set of electrodes). The systems can also be programmed to deliver neuromodulation signals via an existing administration pattern and/or a juggling pattern. In some embodiments, combining multiple therapies is expected to provide one or more advantages, described below.

It has been demonstrated that existing high frequency neuromodulation signals having a frequency in a frequency range of 1.2 kHz to 100 KHz can exhibit a “wash-in” period when administered to treat pain in a patient. The wash-in period refers to the period of time between the onset of signal administration and the onset of therapeutic relief (e.g., pain relief) provided by the signal. This wash-in period can last for a period of time extending from several hours to several days. This wash-in period can be disadvantageous because (1) patients can continue to suffer from pain during this period, and (2) it can make it more challenging to identify which patients respond best to the therapy (e.g., a healthcare provider may need to wait for several days after initial administration to determine if the therapy will be effective in any given patient). Of course, existing high frequency neuromodulation nevertheless provides many benefits, such as those described in U.S. Pat. No. 8,712,533, previously incorporated by reference herein, that make it an attractive therapy option for many patients despite the delayed onset of pain relief.

The modified high frequency neuromodulation signals described herein (e.g., the asymmetric signals described with respect to FIGS. 4A-4C, the signals having relatively long interphase intervals described with respect to FIGS. 5A-5C, and signals delivered in accordance with a juggling pattern as described with respect to FIGS. 6A-6C) can exhibit a more rapid onset of pain relief (e.g., within seconds, within minutes, within hours, etc.) and thus may not be subject to the foregoing disadvantages. For example, FIG. 10 is a graph 1000 illustrating Von Frey paw withdrawal thresholds for rats subjected to various therapies described herein. In particular, the x-axis measures time and the y-axis measures the Von Frey paw withdrawal threshold in grams. For the x-axis, the “baseline” data set was obtained by performing the Von Frey paw withdrawal test on healthy rats, the “prestim” data set was obtained by performing the Von Frey paw withdrawal test following surgical intervention to sensitize the rats, and the “day 1”, “day 3”, and “day 7” data sets were obtained by performing the Von Frey paw withdrawal test after the sensitized rat received stimulation for one day, three days, and 7 days, respectively. Line 1001 illustrates results for rats receiving existing 10 KHz stimulation comprising symmetric bi-phasic pulses, line 1002 illustrates results for rats receiving 5 kHz stimulation with a relatively long interphase interval (e.g., the signals 500a-500c described with respect to FIGS. 5A-5C), and line 1003 illustrates results for rats receiving 5 kHz stimulation with asymmetric pulses (e.g., the signals 400a-400c described with respect to FIGS. 4A-4C). More specifically, the 5 kHz stimulation with the relatively long interphase interval (line 1002) had a leading pulse phase pulse width of 20 microseconds, an interphase interval of 60 microseconds, and a charge balancing phase pulse width of 20 microseconds. Similarly, the 5 kHz asymmetric stimulation (line 1003) had a leading pulse phase pulse width of 20 microseconds and a charge balancing phase pulse width of 60 microseconds.

As shown in FIG. 10, both the 5 kHz long interphase interval stimulation (line 1002) and the 5 kHz asymmetric stimulation (line 1003) resulted in greater pain relief than the existing 10 KHz stimulation (line 1001) at days 1, 3, and 7, as measured by the rat being able to withstand a greater stimulus before the paw withdrawal reflex was activated. In other words, the 5 kHz long interphase interval stimulation and the 5 kHz asymmetric stimulation both demonstrated a more rapid onset of pain relief that was greater in magnitude than pain relief achieved with existing signals for at least the first week of stimulation. Without intending to be bound by theory, this may be in part a result of the modified high frequency neuromodulation signals directly activating excitatory neurons, which, once activated, can activate neighbor inhibitory interneurons not reachable by existing symmetric waveforms because existing symmetric waveforms preferentially activate inhibitory neurons proximate the lead and therefore do not trigger network effects. The activated neighbor inhibitory interneurons could drive inhibition of the second order projection neurons that may be at least partially responsible for the patient's pain. Accordingly, the foregoing signals can be delivered to a patient in combination with existing high frequency neuromodulation to (1) provide a more rapid onset of pain relief, and/or (2) assist in more rapidly identifying which patients respond to high frequency neuromodulation.

FIG. 11 illustrates a graph 1100 illustrating the potential therapeutic effect of combining different types of high frequency neuromodulation signals. Line 1101 illustrates the expected level of pain relief over time for an existing high frequency neuromodulation signal. Line 1102 illustrates the expected level of pain relief over time for a modified high frequency neuromodulation signal (e.g., an asymmetric signal, a signal having a relatively long interphase interval, and/or a signal administered according to a juggling pattern). Line 1103 illustrates the expected level of pain relief over time if an existing high frequency neuromodulation signal is delivered in combination with a modified neuromodulation signal. As illustrated, the existing high frequency neuromodulation signal is expected to demonstrate a delayed onset of pain relief, but, after the initial wash in period, is expected to provide prolonged and robust pain relief. In contrast, the modified high frequency neuromodulation signal is expected to demonstrate a relatively rapid onset of pain relief, but, in certain patients, the pain relief may fall off after an initial period (e.g., due to adaptation of the modulated neurons, as described in Section 6.0). However, the combined effect of the existing high frequency neuromodulation signal is expected to provide both relatively rapid onset of pain relief (from the modified high frequency neuromodulation signal) and prolonged pain relief (from the existing high frequency neuromodulation signal). In some patients, the modified high frequency neuromodulation signal may provide a relatively rapid onset of pain relief that persists for a prolonged period, and therefore may not need to be combined with an existing high frequency neuromodulation signal.

In addition to the foregoing benefit, delivering one or more of the modified signals described herein with existing high frequency signals can help identify if a patient is more likely to respond to high frequency therapy. As previously described, it can take several hours to several days for a patient to have a therapeutic effect from existing high frequency neuromodulation. In contrast, the modified high frequency neuromodulation signals described herein can induce a relatively rapid (e.g., less than several hours, less than several minutes, simultaneous, etc.) onset of pain relief. The patient's response to the modified high frequency neuromodulation signals can therefore be used to project whether the patient will respond to existing high frequency signals. This can decrease time and costs, since an entire trial procedure could be conducted during a single patient visit to a healthcare provider.

In some embodiments, systems in accordance with the present technology can be programmed to simultaneously generate and/or deliver existing high frequency signals and modified high frequency signals (e.g., modified high frequency signals having asymmetric bi-phasic pulses, modified high frequency signals having relatively long interphase intervals, and/or mono-phasic high frequency pulses administered via a juggling pattern). For example, the systems can deliver a modified high frequency signal to provide initial pain relief and/or determine whether the patient responds to high frequency neurostimulation. At the same time, the systems can also deliver an existing high frequency signal to initiate the wash-in period.

In some embodiments, systems in accordance with the present technology can be programmed to generate and/or deliver a modified high frequency signal (e.g., modified high frequency signals having asymmetric bi-phasic pulses, modified high frequency signals having relatively long interphase intervals, and/or mono-phasic high frequency pulses administered via a juggling pattern) before generating and/or delivering an existing high frequency signal. For example, the systems can deliver a modified high frequency signal to provide initial pain relief and/or determine whether the patient responds to high frequency neurostimulation. If it is determined that the patient responds to high frequency neurostimulation, the system can then initiate delivery of the existing high frequency signal. In some embodiments, the delivery of the modified and existing high frequency signals overlap. In other embodiments, the delivery of the modified high frequency signals is terminated before delivering the existing high frequency signals.

In some embodiments, systems in accordance with the present technology can deliver a modified high frequency signal (e.g., modified high frequency signals having asymmetric bi-phasic pulses, modified high frequency signals having relatively long interphase intervals, and/or mono-phasic high frequency pulses administered via a juggling pattern) to one or more electrodes implanted in a patient at a first position having a relatively high perception threshold and deliver an existing high frequency signal to one or more electrodes implanted in the patient at a second position having a relatively low perception threshold. In some embodiments, the modified high frequency signals may require less energy than existing (e.g., symmetric) high frequency signals for a given amplitude. Accordingly, the modified high frequency signals can be administered to the first position having the relatively high perception threshold at a relatively high amplitude without substantially draining the system's power source.

In some embodiments, systems in accordance with the present technology can be programmed to generate both modified and existing high frequency signals. A user (e.g., the patient, a healthcare provider, etc.) can selectively activate the modified high frequency signal, the existing high frequency signal, or both the modified and existing high frequency signal. The user can select which signal to deliver using a user interface on a controller operably coupled to the signal generator.

### 8.0 Additional Expected Benefits Associated with Certain Embodiments

The foregoing embodiments can produce one or more of a variety of advantages, for the patient and/or practitioner, when compared with existing SCS therapy. Some of these benefits were described above. For example, the modified high frequency neuromodulation signals described herein can induce a more rapid onset of pain relief in a patient, which is beneficial to both the patient (faster pain relief) and the practitioner (easier to identify patient responders). In addition, the modified high frequency neuromodulation signals can modulate a target population of neurons in a different manner than existing high frequency neuromodulation signals, and thus can provide a supplementary and/or synergistic effect when delivered with existing high frequency neuromodulation.

In addition to the foregoing, the modified high frequency signals described herein can induce effective pain relief without patient-detectable disruptions to normal sensory and motor signals along the spinal cord. While the therapy may create some effect on normal motor and/or sensory signals, the effect is below a level that the patient can reliably detect intrinsically, e.g., without the aid of external assistance via instruments or other devices. Accordingly, the patient's levels of motor signaling and other sensory signaling (other than signaling associated with the target pain) can be maintained at pre-treatment levels. For example, the patient can experience a significant pain reduction that is largely independent of the patient's movement and position. In particular, the patient can assume a variety of positions and/or undertake a variety of movements associated with activities of daily living and/or other activities, without the need to adjust the parameters in accordance with which the therapy is applied to the patient (e.g., the signal amplitude). This result can greatly simplify the patient's life and reduce the effort required by the patient to experience pain relief while engaging in a variety of activities. This result can also provide an improved lifestyle for patients who experience pain during sleep.

Another expected benefit is that, similar to existing high frequency neuromodulation signals, the modified high frequency signals described herein are expected to produce pain relief in the patient without generating paresthesia. In contrast, existing low frequency neuromodulation (e.g., signals with frequencies less than 1.2 kHz) typically are commonly understood to rely on paresthesia (i.e., masking) to provide a therapeutic effect (i.e., pain relief). However, although described in the context of high frequency signals, the waveforms and administration patterns described herein could also be applied to low frequency signals, such as those having frequencies less than 1.2 kHz.

### 9.0 Representative Examples

The following examples are provided to further illustrate embodiments of the present technology and are not to be interpreted as limiting the scope of the present technology. To the extent that certain embodiments or features thereof are mentioned, it is merely for purposes of illustration and, unless otherwise specified, is not intended to limit the present technology. It will be understood that many variations can be made in the procedures described herein while still remaining within the bounds of the present technology. Such variations are intended to be included within the scope of the presently disclosed technology.

1. A patient treatment system, comprising:
a signal generator having a computer readable storage medium, wherein, in operation, the signal generator generates an electrical signal having bi-phasic pulses repeating at a frequency within a frequency range of about 1.2 kHz to about 500 kHz, wherein each bi-phasic pulse includes a first pulse phase having a first pulse width and a first polarity, a second pulse phase having a second pulse width and a second polarity opposite the first polarity, and an interphase interval between the first pulse phase and the second pulse phase, and wherein:
   (a) a ratio of the second pulse phase pulse width to the first pulse phase pulse width is between 2:1 and 6:1, or
   (b) a ratio of a duration of the of the interphase interval to the first pulse phase pulse width is between 2:1 and 6:1, or
   (c) both (a) and (b); and
a signal delivery element coupleable to the signal generator, wherein the signal delivery element is positionable proximate a target region, and, in operation, delivers the electrical signal to the target region.

2. The system of example 1 wherein the ratio of the second pulse phase pulse width to the first pulse phase pulse width is between 2:1 and 6:1.

3. The system of example 2 wherein the ratio of the second pulse phase pulse width to the first pulse phase pulse width is between 2:1 and 5:1.

4. The system of example 2 wherein the ratio of the second pulse phase pulse width to the first pulse phase pulse width is between 2:1 and 4:1.

5. The system of example 2 wherein the ratio of the second pulse phase pulse width to the first pulse phase pulse width is about 3:1.

6. The system of example 2 wherein the first pulse phase pulse width is in a pulse width range of about 1 microsecond to about 416 microseconds, and wherein the second pulse phase pulse width is in a pulse width range of about 2 microseconds to about 740 microseconds.

7. The system of any of examples 2-6 wherein the first pulse phase has a first amplitude and the second pulse phase has a second amplitude, and wherein the ratio between the second amplitude and the first amplitude is between 1:2 and 1:6.

8. The system of example 7 wherein the ratio between the second pulse phase amplitude and the first pulse phase amplitude is the inverse of the ratio between the second pulse phase pulse width and the first pulse phase pulse width such that the total charge delivered during the first pulse phase and the total charge delivered during the second pulse phase are equal and opposite.

9. The system of example 1 wherein the ratio of the duration of the interphase interval to the first pulse phase pulse width is between 2:1 and 6:1.

10. The system of example 9 wherein the ratio of the duration of the interphase interval to the first pulse phase pulse width is between 2:1 and 5:1.

11. The system of example 9 wherein the ratio of the duration of the interphase interval to the first pulse phase pulse width is between 2:1 and 4:1.

12. The system of example 9 wherein the ratio of the duration of the interphase interval to the first pulse phase pulse width is about 3:1.

13. The system of any of examples 9-12 wherein the duration of the interphase interval is between about 24 microseconds and about 160 microseconds.

14. The system of any of examples 9-12 wherein the duration of the interphase interval is between about 40 microseconds and about 80 microseconds.

15. The system of any of examples 1-14 wherein both (a) the ratio of the second pulse phase pulse width to the first pulse phase pulse width is between 2:1 and 6:1, and (b) the ratio of the duration of the interphase interval to the first pulse phase pulse width is between 2:1 and 6:1.

16. The system of any of examples 1-15 wherein the first pulse phase is an anodic pulse phase and the second pulse phase is a cathodic pulse phase.

17. The system of any of examples 1-15 wherein the first pulse phase is a cathodic pulse phase and the second pulse phase is an anodic pulse phase.

18. The system of any of examples 1-17 wherein the first pulse phase is a therapy pulse phase and the second pulse phase is a charge balancing pulse phase.

19. The system of any of examples 1-18 wherein the frequency is in a frequency range of from about 1.2 kHz to about 50 KHz.

20. The system of any of examples 1-18 wherein the frequency is in a frequency range of from about 3 kHz to about 15 kHz.

21. The system of any of examples 1-20 wherein the electrical signal is a first electrical signal, and wherein, in operation, the signal generator further generates a second electrical signal having bi-phasic pulses repeating at a frequency within a frequency range of about 1.2 kHz to about 500 kHz, wherein the bi-phasic pulses of the second electrical signal have a third pulse phase having a third pulse width and a third polarity, a fourth pulse phase having a fourth pulse width and a fourth polarity opposite the third polarity, and an interphase interval between the third pulse phase and the second pulse phase, and wherein:
(a) the third pulse phase pulse width is approximately equal to the fourth pulse phase pulse width; and
(b) the duration of the interphase interval is less than or equal to the third pulse phase pulse width.

22. The system of example 21 wherein, in operation, the system generates and delivers the first electrical signal and the second electrical signal simultaneously.

23. The system of example 21 wherein, in operation, the system generates and delivers the first electrical signal and the second electrical signal sequentially.

24. A method of treating a patient, comprising:
programming a signal generator to generate an electrical signal having bi-phasic pulses repeating at a frequency within a frequency range of about 1.2 kHz to about 500 kHz, wherein each bi-phasic pulse includes a first pulse phase having a first pulse width and a first polarity, a second pulse phase having a second pulse width and a second polarity opposite the first polarity, and an interphase interval between the first pulse phase and the second pulse phase, and wherein:
   (a) a ratio of the second pulse phase pulse width to the first pulse phase pulse width is between 2:1 and 6:1, or
   (b) a ratio of a duration of the of the interphase interval to the first pulse phase pulse width is between 2:1 and 6:1, or
   (c) both (a) and (b).

25. The method of example 24 wherein the ratio of the second pulse phase pulse width to the first pulse phase pulse width is between 2:1 and 6:1.

26. The method of example 25 wherein the ratio of the second pulse phase pulse width to the first pulse phase pulse width is between 2:1 and 5:1.

27. The method of example 25 wherein the ratio of the second pulse phase pulse width to the first pulse phase pulse width is between 2:1 and 4:1.

28. The method of example 25 wherein the ratio of the second pulse phase pulse width to the first pulse phase pulse width is about 3:1.

29. The method of example 25 wherein the first pulse phase pulse width is in a pulse width range of about 1 microsecond to about 416 microseconds, and wherein the second pulse phase pulse width is in a pulse width range of about 2 microseconds to about 740 microseconds.

30. The method of any of examples 25-29 wherein the first pulse phase has a first amplitude and the second pulse phase has a second amplitude, and wherein the ratio between the second amplitude and the first amplitude is between 1:2 and 1:6.

31. The method of example 30 wherein the ratio between the second pulse phase amplitude and the first pulse phase amplitude is the inverse of the ratio between the second pulse phase pulse width and the first pulse phase pulse width such that the total charge delivered during the first pulse phase and the total charge delivered during the second pulse phase are equal and opposite.

32. The method of example 24 wherein the ratio of the duration of the interphase interval to the first pulse phase pulse width is between 2:1 and 6:1.

33. The method of example 32 wherein the ratio of the duration of the interphase interval to the first pulse phase pulse width is between 2:1 and 5:1.

34. The method of example 32 wherein the ratio of the duration of the interphase interval to the first pulse phase pulse width is between 2:1 and 4:1.

35. The method of example 32 wherein the ratio of the duration of the interphase interval to the first pulse phase pulse width is about 3:1.

36. The method of any of examples 32-35 wherein the duration of the interphase interval is between about 24 microseconds and about 160 microseconds.

37. The method of any of examples 32-35 wherein the duration of the interphase interval is between about 40 microseconds and about 80 microseconds.

38. The method of any of examples 24-37 wherein both (a) the ratio of the second pulse phase pulse width to the first pulse phase pulse width is between 2:1 and 6:1, and (b) the ratio of a duration of the of the interphase interval to the first pulse phase pulse width is between 2:1 and 6:1.

39. The method of any of examples 24-38 wherein the first pulse phase is an anodic pulse phase and the second pulse phase is a cathodic pulse phase.

40. The method of any of examples 24-38 wherein the first pulse phase is a cathodic pulse phase and the second pulse phase is an anodic pulse phase.

41. The method of any of examples 24-40 wherein the first pulse phase is a therapy pulse phase and the second pulse phase is a charge balancing pulse phase.

42. The method of any of examples 24-41 wherein the frequency is in a frequency range of from about 1.2 kHz to about 50 KHz.

43. The method of any of examples 24-41 wherein the frequency is in a frequency range of from about 3 kHz to about 15 kHz.

44. The method of any of examples 24-43 wherein the electrical signal is a first electrical signal, the method further comprising:
programming the signal generator to generate a second electrical signal having bi-phasic pulses repeating at a frequency within a frequency range of about 1.2 kHz to about 500 kHz, wherein the bi-phasic pulses of the second electrical signal have a third pulse phase having a third pulse width and a third polarity, a fourth pulse phase having a fourth pulse width and a fourth polarity opposite the third polarity, and an interphase interval between the third pulse phase and the second pulse phase, and wherein:
   (a) the third pulse phase pulse width is approximately equal to the fourth pulse phase pulse width; and
   (b) the duration of the interphase interval is less than or equal to the third pulse phase pulse width.

45. The method of example 44 wherein programming the signal generator includes programming the signal generator to simultaneously generate the first electrical signal and the second electrical signal.

46. The method of example 44 wherein programming the signal generator includes programming the signal generator to generate the first electrical signal before generating the second electrical signal.

47. The method of any of examples 44-46 wherein the first electrical signal preferentially activates a first set of target neurons, and wherein the second electrical signal preferentially activates a second set of target neurons.

48. The method of example 47 wherein the first set of target neurons includes excitatory neurons and the second set of target neurons includes inhibitory neurons.

49. The method of any of examples 44-48 wherein the first electrical signal induces a first physiological effect, and wherein the second electrical signal induces a second physiological effect different than the first physiological effect.

50. A patient treatment system, comprising:
a signal generator having a computer readable storage medium, wherein, in operation, the signal generator generates an electrical signal having pulses repeating at a frequency within a frequency range of about 1.2 kHz to about 500 KHz; and
a signal delivery element positionable in a target region and having at least three contacts, wherein the signal delivery element is coupleable to the signal generator, and, in operation, delivers the electrical signal to the target region via the at least three contacts,
wherein an individual pulse is delivered via a pair of contacts that is different than the pair of contacts used to deliver the immediately preceding pulse.

51. The system of example 50 wherein (a) the at least three contacts includes a first contact, a second contact, and a third contact, and (b) the pulses include a first pulse, a second pulse, and a third pulse, and wherein, in operation:
the first pulse is delivered via the first and second contacts,
the second pulse is delivered via the second and third contacts, and
the third pulse is delivered via the third and first contacts.

52. The system of example 51 wherein:
during delivery of the first pulse, current flows out of the first contact and into the second contact,
during delivery of the second pulse, current flows out of the second contact and into the third contact, and
during delivery of the third pulse, current flows out of the third contact and into the first contact.

53. The system of examples 51 or 52 wherein the first pulse, the second pulse, and the third pulse have the same amplitude and pulse width.

54. The system of examples 51 or 52 wherein at least one of the first pulse, the second pulse, and the third pulse has an amplitude and/or pulse width that is different than an amplitude and/or pulsed width of at least one of the other of the first pulse, the second pulse, or the third pulse.

55. The system of example 54 wherein the total charge delivered by the first pulse, the second pulse, and the third pulse is the same.

56. The system of any of examples 50-55 wherein the signal delivery element includes more than three contacts.

57. The system of any of examples 50-56 wherein the frequency is in a frequency range of from about 1.2 kHz to about 50 KHz.

58. The system of any of examples 50-56 wherein the frequency is in a frequency range of from about 3 kHz to about 15 kHz.

59. A method of treating a patient, comprising:
programming a patient treatment system having a signal generator and a signal delivery element to:
   generate, via the signal generator, an electrical signal having pulses repeating at a frequency within a frequency range of about 1.2 kHz to about 500 kHz; and
   deliver, via the signal delivery element, the electrical signal to the target region,
   wherein the signal delivery element includes at least three contacts, and wherein the system is programmed such that an individual pulse is delivered via a pair of contacts that is different than the pair of contacts used to deliver the immediately preceding pulse.

60. The method of example 59 wherein (a) the at least three contacts include a first contact, a second contact, and a third contact, and (b) the pulses include a first pulse, a second pulse, and a third pulse and wherein programming the patient treatment system includes programing the patient treatment system such that:
the first pulse is delivered via the first and second contacts,
the second pulse is delivered via the second and third contacts, and
the third pulse is delivered via the third and first contacts.

61. The method of example 60 wherein programming the patient treatment system includes programming the patient treatment system such that:
during delivery of the first pulse, current flows out of the first contact and into the second contact,
during delivery of the second pulse, current flows out of the second contact and into the third contact, and
during delivery of the third pulse, current flows out of the third contact and into the first contact.

62. The method of examples 60 or 61 wherein the first pulse, the second pulse, and the third pulse have the same amplitude and pulse width.

63. The method of examples 60 or 61 wherein at least one of the first pulse, the second pulse, and the third pulse has an amplitude and/or pulse width that is different than an amplitude and/or pulsed width of at least one of the other of the first pulse, the second pulse, or the third pulse.

64. The method of example 63 wherein the total charge delivered by the first pulse, the second pulse, and the third pulse is the same.

65. The method of any of examples 59-64 wherein the signal delivery element includes more than three contacts.

66. The method of any of examples 59-65 wherein the frequency is in a frequency range of from about 1.2 kHz to about 50 KHz.

67. The method of any of examples 59-65 wherein the frequency is in a frequency range of from about 3 kHz to about 15 KHz.

68. The method of any of examples 59-67 wherein the electrical signal preferentially modulates a first set of target neurons when administered at a first amplitude, and wherein the electrical signal preferentially modulates a second set of target neurons different than the first set when administered at a second amplitude.

69. The method of example 68 wherein the first amplitude is greater than or equal to 30 percent of motor threshold, and wherein the first set of target neurons includes excitatory neurons.

70. The method of example 68 or 69 wherein the second amplitude is less than or equal to 10 percent of motor threshold, and wherein the second set of target neurons includes inhibitory neurons.

### 10.0 Conclusion

From the foregoing, it will be appreciated that specific embodiments of the disclosed technology have been described herein for purposes of illustration, but that various modifications can be made without deviating from the technology. For example, therapy signals described herein can be delivered at combinations of parameter values within the foregoing ranges at values that are not expressly disclosed herein. Certain aspects of the technology described in the context of particular embodiments can be combined or eliminated in other embodiments. In some embodiments, the foregoing techniques can be used to address patient deficits than pain. Further, while advantages associated with certain embodiments of the disclosed technology have been described in the context of those embodiments, other embodiments may also exhibit such advantages, and not all embodiments need necessarily exhibit such advantages to fall within the scope of the present technology. Accordingly, the disclosure and associated technology can encompass other embodiments not expressly shown or described herein.

The use of “and/or” in reference to a list of two or more items is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications can be made without deviating from the technology. Further, while advantages associated with certain embodiments of the technology have been described in the context of those embodiments, other embodiments can also exhibit such advantages, and not all embodiments need necessarily exhibit such advantages to fall within the scope of the technology. Accordingly, the disclosure and associated technology can encompass other embodiments not expressly shown or described herein.

Where a range of values is provided, it is understood that each intervening value, to the tenth of the unit of the lower limit unless the context clearly dictates otherwise, to between the upper and lower limits of that range is also specifically disclosed. Each smaller range between any stated value or intervening value in a stated range and any other stated or intervening value in that stated range is encompassed within the disclosure. The upper and lower limits of these smaller ranges can independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

To the extent any materials incorporated herein by reference conflict with the present disclosure, the present disclosure controls.

I/We claim:

1. A patient treatment system, comprising:
a signal generator having a computer readable storage medium, wherein, in operation, the signal generator generates an electrical signal having pulses repeating at a frequency within a frequency range of about 1.2 kHz to about 500 KHz; and
a signal delivery element positionable in a target region and having at least three contacts, wherein the signal delivery element is coupleable to the signal generator, and, in operation, delivers the electrical signal to the target region via the at least three contacts,
wherein an individual pulse is delivered via a pair of contacts that is different than the pair of contacts used to deliver the immediately preceding pulse.

2. The system of claim 1 wherein (a) the at least three contacts includes a first contact, a second contact, and a third contact, and (b) the pulses include a first pulse, a second pulse, and a third pulse, and wherein, in operation:
the first pulse is delivered via the first and second contacts,
the second pulse is delivered via the second and third contacts, and
the third pulse is delivered via the third and first contacts.

3. The system of claim 2 wherein:
during delivery of the first pulse, current flows out of the first contact and into the second contact,
during delivery of the second pulse, current flows out of the second contact and into the third contact, and
during delivery of the third pulse, current flows out of the third contact and into the first contact.

4. The system of claim 2 wherein the first pulse, the second pulse, and the third pulse have the same amplitude and pulse width.

5. The system of claim 2 wherein at least one of the first pulse, the second pulse, and the third pulse has an amplitude and/or pulse width that is different than an amplitude and/or pulsed width of at least one of the other of the first pulse, the second pulse, or the third pulse.

6. The system of claim 5 wherein the total charge delivered by the first pulse, the second pulse, and the third pulse is the same.

7. The system of claim 1 wherein the signal delivery element includes more than three contacts.

8. The system of claim 1 wherein the frequency is in a frequency range of from about 1.2 kHz to about 50 KHz.

9. The system of claim 1 wherein the frequency is in a frequency range of from about 3 kHz to about 15 KHz.

10. A method of treating a patient, comprising:
programming a patient treatment system having a signal generator and a signal delivery element to:
generate, via the signal generator, an electrical signal having pulses repeating at a frequency within a frequency range of about 1.2 kHz to about 500 KHz; and
deliver, via the signal delivery element, the electrical signal to the target region,
wherein the signal delivery element includes at least three contacts, and wherein the system is programmed such that an individual pulse is delivered via a pair of contacts that is different than the pair of contacts used to deliver the immediately preceding pulse.

11. The method of claim 10 wherein (a) the at least three contacts include a first contact, a second contact, and a third contact, and (b) the pulses include a first pulse, a second pulse, and a third pulse and wherein programming the patient treatment system includes programing the patient treatment system such that:
the first pulse is delivered via the first and second contacts,
the second pulse is delivered via the second and third contacts, and
the third pulse is delivered via the third and first contacts.

12. The method of claim 11 wherein programming the patient treatment system includes programming the patient treatment system such that:
during delivery of the first pulse, current flows out of the first contact and into the second contact,
during delivery of the second pulse, current flows out of the second contact and into the third contact, and
during delivery of the third pulse, current flows out of the third contact and into the first contact.

13. The method of claim 11 wherein the first pulse, the second pulse, and the third pulse have the same amplitude and pulse width.

14. The method of claim 11 wherein at least one of the first pulse, the second pulse, and the third pulse has an amplitude and/or pulse width that is different than an amplitude and/or pulsed width of at least one of the other of the first pulse, the second pulse, or the third pulse.

15. The method of claim 14 wherein the total charge delivered by the first pulse, the second pulse, and the third pulse is the same.

16. The method of claim 10 wherein the signal delivery element includes more than three contacts.

17. The method of claim 10 wherein the frequency is in a frequency range of from about 1.2 kHz to about 50 KHz.

18. The method of claim 10 wherein the frequency is in a frequency range of from about 3 kHz to about 15 kHz.

19. The method of a claim 10 wherein the electrical signal preferentially modulates a first set of target neurons when administered at a first amplitude, and wherein the electrical signal preferentially modulates a second set of target neurons different than the first set when administered at a second amplitude.

20. The method of claim 19 wherein the first amplitude is greater than or equal to 30 percent of motor threshold, and wherein the first set of target neurons includes excitatory neurons.
